SEQUENCE LISTING (7 sequences)

SEQ ID NO: 1 - protein, 194 aa, Artificial Sequence
  SARS-CoV-2 Spike Protein Native RBD
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG

SEQ ID NO: 2 - protein, 194 aa, Artificial Sequence
  Recombinant RBD Immunogen, Lead 1, Decoy 28
MNLCPFGEVFNATRFPSVYAWNRKRISNCYYDYSVLYNSASFSTFKCYGVSPTKLNDLCFTQVFADSFVIRGDEVRQIAPGQTGKIADYNYKLPDDFTGCVIAWNSNNLDSKVGGNYNYLYRLFRKSNLKPFERDTSTEIYQAGSTPCNGVEGFNCYFPLQSYGFQPTNGVGYQPYRVVVLSFELLDAPPTVCG

SEQ ID NO: 3 - protein, 194 aa, Artificial Sequence
  Recombinant RBD Immunogen, Lead 2, Decoy 24
TNLCPFGEVFNATRFASVYAWNRKRISNCQFDYSKLYNSASFSTFKCYGVSPTKLNDLCFTQVYADSFVIRGDEVRQIAPGQTGKIADYNYKLPDDFTGCVIAWNSNNLDSKVGGNYNYLYRLFRKSNLKPFERDTSTEIYQAGSTPCNGVEGFNCYFPLQSYGFQPTNGVGYQPYRVVVLTFELLDAPATVCG

SEQ ID NO: 4 - protein, 194 aa, Artificial Sequence
  Recombinant RBD Immunogen, Lead 3, Decoy 25
KNLCPFGEVFNATRFASVYAWNRKRISNCVYDYSVLYNSASFSTFKCYGVSPTKLKDLCFTYVYADSFVIRGDEVRQIAPGQTGKIADYNYKLPDDFTGCVIAWNSNNLDSKVGGNYNYLYRLFRKSNLKPFERDTSTEIYQAGSTPCNGVEGFNCYFPLQSYGFQPTNGVGYQPYRVVVLTFELLDAPPTVCG

SEQ ID NO: 5 - protein, 194 aa, Artificial Sequence
  Recombinant RBD Immunogen, Lead 4, Decoy 26
TNLCPFGEVFNATRFASVYAWNRKRISNCKYDYSALYNSASFSTFKCYGVSPTKLNDLCFTEVYADYFVVRGDEVRQIAPGQTGKIADYNYKLPDDFTGCVIAWNSNNLDSKVGGNYNYLYRLFRKSNLKPFERDTSTEIYQAGSTPCNGVEGFNCYFPLQSYGFQPTNGVGYQPYRVVVLTFELKDAPATVCG

SEQ ID NO: 6 - protein, 194 aa, Artificial Sequence
  Recombinant RBD Immunogen, Lead 5, Decoy 21
TNLCPFGEVFNATRFPAVYAWNRKRISNCVFDFSKLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLQPFERDTSEEIYQAGSTPCNGVEGFNCYFPLQSYGFQPTNGVGYQPYRVVVLNFELLDAPPTVCG

SEQ ID NO: 7 - protein, 194 aa, Artificial Sequence
  Non-glycosylated RBD
TNPCPFEEIFHAERFASVYAWQRKQISDCVWDFSLLLNSKSFSTFKCYGVSPKQLNDLCFTNVYADSFVIRGDEVQQIAPGQTGKIADYNYKLPDDFTGCVIAWNSNDLDSKVGGNYDYLYRLFRKDKLKPYERDTSTEIYQAGDTPCNGVEGFNCYNPLQSYGFQPTNGVGYQPYRVVVLTFQLLDAPATVCG

(19) United States
(12) Patent Application Publication
(10) Pub. No.: US 2025/0281600 A1
(43) Pub. Date: Sep. 11, 2025

(54) NOVEL COMPOSITIONS OF MATTER COMPRISING STABILIZED CORONAVIRUS ANTIGENS AND THEIR USE

(71) Applicant: The United States of America, as represented by the Secretary, Department of Health and Human, Bethesda, MD (US)

(72) Inventors: Niraj Harish Tolia, Bethesda, MD (US); Thayne Henderson Dickey, Bethesda, MD (US)

(21) Appl. No.: 18/277,921

(22) PCT Filed: Feb. 18, 2022
(86) PCT No.: PCT/US2022/070744

§ 371 Date: Aug. 18, 2023

Related U.S. Application Data
(60) Provisional application No. 63/200,194, filed on Feb. 19, 2021.

(51) Int. Cl. A61K 39/215; A61K 39/00; A61P 31/14; C07K 14/005; C12N 7/00; G16B 15/30; G16B 40/30

(52) U.S. Cl. CPC A61K 39/215; A61P 31/14; C07K 14/005; C12N 7/00; G16B 15/30; G16B 40/30; A61K 2039/55555; C12N 2770/20022

(57) ABSTRACT

New compositions of matter, suitable as vaccine candidates, were derived from target antigens using a novel computational design pipeline and in vitro screening. The new compositions of matter include new protein sequences derived from the receptor binding domain (RBD) of a coronavirus spike protein. The present disclosure provides immunological compositions and methods related to the design, production, and administration of such compositions.

### CROSS-REFERENCE TO RELATED APPLICATIONS

This application claims the benefit of priority to U.S. Prov. Pat. App. No. 63/200,194 filed Feb. 19, 2021, which is incorporated by reference herein in its entirety.

### STATEMENT REGARDING FEDERALLY FUNDED RESEARCH & DEVELOPMENT

This invention was made in part with government support from the National Institutes of Health. The government has certain rights in this invention.

### SEQUENCE LISTING

This application contains a sequence listing in paper format and in computer readable format, the teachings and content of which are hereby incorporated by reference.

### BACKGROUND OF THE DISCLOSURE

An effective and easily distributed vaccine that confers long-lasting protection against SARS-COV-2 is required to combat the global COVID-19 epidemic. Two vaccines have been granted emergency use authorization by the FDA to date, and several others are in advanced clinical trials. Nearly all of these vaccines contain an engineered SARS-COV-2 spike protein antigen with two stabilizing proline mutations in the S2 domain. These mutations improve the efficacy of coronavirus vaccines, but they are limited in their focus on the S2 domain. Stabilizing mutations in other spike protein domains may further improve vaccine characteristics by increasing SARS-COV-2 vaccine efficacy, addressing distribution challenges associated with current vaccines, and informing the design of vaccines to address future coronavirus outbreaks.

The receptor binding domain (RBD) of the spike protein is a second antigen used in vaccine candidates (FIG. 1B). The RBD has several advantages over the full-length protein. Multiple studies have shown that the most potent SARS-COV2 neutralizing antibodies bind the RBD and block its interaction with the human receptor Ace2. However, these potent neutralizing antibodies are extremely rare relative to non-neutralizing antibodies that bind epitopes elsewhere on the spike protein. Therefore, immunization with an RBD antigen would be expected to focus the immune response to neutralizing epitopes and avoid the production of non-neutralizing potentially pathogenic antibodies against FL-spike.

Production and use of an RBD antigen is complicated by the fact that it is glycosylated. The RBD (residues 333-527) contains an N-linked glycosylation site at position 343 where large heterogeneous sugars are added post-translationally. The size and composition of these glycans depends on many factors, including cell type used for expression. These variable factors mean that glycoprotein characteristics can vary between expression platforms and between batches. Variability in glycan composition can have a major impact on the immunogenicity, stability, solubility, and efficacy of a vaccine, leading to challenges associated with the production of homogenous reproducible glycoprotein vaccines. Conventional vaccines are typically heavily glycosylated.

### SUMMARY OF THE DISCLOSURE

An aspect of the present disclosure provides a protein having at least 90% sequence homology with a sequence selected from the group consisting of SEQ ID NO: 2, SEQ ID NO: 3, SEQ ID NO: 4, SEQ ID NO: 5, and SEQ ID NO: 6.

Another aspect of the disclosure provides a composition comprising: at least one protein having at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof; and a pharmaceutically-acceptable carrier selected from the group consisting of a solvent, a dispersion media, a coating, a stabilizing agent, a diluent, a preservative, an antimicrobial agent, an antifungal agent, an isotonic agent, and an adsorption delaying agent.

In some embodiments, the at least one protein is a receptor binding domain (RBD) recombinant protein, the RBD is associated with at least one variant of SARS-COV-2 virus, the composition further comprises an immune stimulant, and/or the at least one protein has at least 95%, at least 96%, at least 97%, at least 98%, or at least 99% sequence homology.

Yet another aspect of the disclosure provides a nucleotide sequence encoding a protein, wherein the protein has at least 90% sequence homology with a sequence selected from the group consisting of SEQ ID NO: 2, SEQ ID NO: 3, SEQ ID NO: 4, SEQ ID NO: 5, and SEQ ID NO: 6.

A further aspect of the disclosure provides a composition comprising: at least one nucleotide sequence encoding a respective protein, wherein the respective protein has at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof; and a pharmaceutically-acceptable carrier selected from the group consisting of a solvent, a dispersion media, a coating, a stabilizing agent, a diluent, a preservative, an antimicrobial agent, an antifungal agent, an isotonic agent, and an adsorption delaying agent.

Another aspect of the disclosure provides at least a partial coronavirus spike protein comprising a receptor binding domain (RBD), wherein the RBD has at least 90% sequence homology with a sequence selected from the group consisting of SEQ ID NO: 2, SEQ ID NO: 3, SEQ ID NO: 4, SEQ ID NO: 5, and SEQ ID NO: 6.

Yet another aspect of the disclosure provides a composition comprising: at least one partial coronavirus spike protein comprising a receptor binding domain (RBD), wherein the RBD has at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof; and a pharmaceutically-acceptable carrier selected from the group consisting of a solvent, a dispersion media, a coating, a stabilizing agent, a diluent, a preservative, an antimicrobial agent, an antifungal agent, an isotonic agent, and an adsorption delaying agent.

In some embodiments, the coronavirus spike protein is a SARS-COV-2 spike protein, the at least one partial coronavirus spike protein comprises at least one full-length coronavirus spike protein, the RBD comprises residues 333-527 of the coronavirus spike protein, and/or the RBD has at least 95%, at least 96%, at least 97%, at least 98%, or at least 99% sequence homology.

A further aspect of the disclosure provides a method for identifying at least one vaccine candidate, the method comprising: determining a computational search depth for each amino acid position of a protein sequence of a target antigen as one of fixed, intermediate, or deep search; performing a computational search for each amino acid position, wherein a fixed depth is defined as leaving a residue of the protein sequence unchanged, wherein an intermediate depth is defined as leaving a residue of the protein sequence unchanged, and wherein a deep search depth is defined as allowing sampling of all amino acids except cysteine; clustering high-scoring decoy sequences resulting from the computational search based on sequence similarity; and screening at least one decoy sequence from each cluster in vitro.

Another aspect of the disclosure provides a method for identifying at least one vaccine candidate, the method comprising: characterizing a computational search depth for each amino acid position of a protein sequence of a target antigen as one of fixed, intermediate, or deep search; performing a computational search for each amino acid position, wherein a fixed depth is defined as leaving a residue of the protein sequence unchanged, wherein an intermediate depth is defined as allowing sampling of amino acids found in proteins with similar sequences based on evolutionary constraints, and wherein a deep search depth is defined as allowing sampling of all amino acids; clustering high-scoring decoy sequences resulting from the computational search based on sequence similarity; and screening at least one decoy sequence from each cluster in vitro.

Yet another aspect of the disclosure provides a method for identifying at least one vaccine candidate, the method comprising: characterizing a computational search depth for each amino acid position of a protein sequence of a target antigen as one of fixed, intermediate, or deep search; performing a computational search for each amino acid position, wherein a fixed depth is defined as leaving a residue of the protein sequence unchanged, wherein an intermediate depth is defined as allowing sampling of all amino acids, and wherein a deep search depth is defined as allowing sampling of all amino acids; clustering high-scoring decoy sequences resulting from the computational search based on sequence similarity; and screening at least one decoy sequence from each cluster in vitro.

A further aspect of the disclosure provides a method is provided for identifying at least one vaccine candidate comprising at least one non-glycosylated RBD recombinant protein, the method comprising: characterizing a computational search depth for each amino acid position of a protein sequence of a target antigen as one of fixed, intermediate, or deep search; performing a computational search for each amino acid position, wherein a fixed depth is defined as leaving a residue of the protein sequence unchanged, wherein an intermediate depth is defined as allowing sampling of amino acids that do not decrease expression of the RBD in a deep mutational scanning experiment, and wherein a deep search depth is defined as allowing sampling of all amino acids; clustering high-scoring decoy sequences resulting from the computational search based on sequence similarity; and screening at least one decoy sequence from each cluster in vitro.

In some embodiments, the target antigen is a receptor binding domain (RBD) of SARS-COV-2. In some embodiments, the deep search depth is defined as allowing sampling of all amino acids except cysteine. In some embodiments, clustering high-scoring decoy sequences comprises clustering decoy sequences with scores in a 90th percentile, a 95th percentile, a 96th percentile, a 97th percentile, a 98th percentile, or a 99th percentile based on sequence similarity. In some embodiments, when a residue of the protein sequence forms a functionally required interface, the amino acid is characterized as fixed. In some embodiments, the functionally required interface is an interface with a receptor. In some embodiments, the receptor is an Ace2 receptor. In some embodiments, when a residue of the protein sequence forms an interface with at least one neutralizing or inhibitory antibody, the amino acid is characterized as fixed. In some embodiments, when a residue of the protein sequence forms an interface with a glycan, the amino acid is characterized as deep search. In some embodiments, when a residue of the protein sequence is exposed in an RBD-up conformation, the amino acid is characterized as deep search. In some embodiments, when a residue of the protein sequence is exposed upon extraction of the target antigen from an associated full-length protein/complex, the amino acid is characterized as deep search. In some embodiments, the target antigen is a receptor binding domain (RBD) of SARS-COV-2, and wherein the associated full-length protein is a full-length spike protein of SARS-COV-2. In some embodiments, the computational search is constrained by disallowing amino acid changes to intermediate and deep search residues that, when made individually, are energetically unfavorable based on energetic constraints. In some embodiments, the computational search is constrained by disallowing amino acid changes to intermediate residues that, when made individually, are energetically unfavorable based on energetic constraints. In some embodiments, the computational search is constrained by disallowing amino acid changes that, when made individually, decrease recombinant RBD expression, as measured by deep mutational scanning.

Another aspect of the disclosure provides a non-glycosylated receptor binding domain (RBD) recombinant protein having at least 85% sequence homology with SEQ ID NO: 7.

Yet another aspect of the disclosure provides a composition comprising: at least one non-glycosylated receptor binding domain (RBD) recombinant protein having at least 85% sequence homology with SEQ ID NO: 7; and a pharmaceutically-acceptable carrier selected from the group consisting of a solvent, a dispersion media, a coating, a stabilizing agent, a diluent, a preservative, an antimicrobial agent, an antifungal agent, an isotonic agent, and an adsorption delaying agent.

In some embodiments, the RBD is associated with at least one variant of SARS-COV-2 virus, the composition further comprises an immune stimulant, and/or the at least one non-glycosylated receptor binding domain (RBD) recombinant protein has at least 90%, at least 95%, at least 97%, or at least 99% sequence homology.

A further aspect of the disclosure provides a nucleotide sequence encoding a non-glycosylated receptor binding domain (RBD) protein, wherein the non-glycosylated RBD protein has at least 85% sequence homology with SEQ ID NO: 7.

Another aspect of the disclosure provides a composition comprising: a nucleotide sequence encoding a non-glycosylated receptor binding domain (RBD) protein, wherein the RBD protein has at least 85% sequence homology with SEQ ID NO: 7; and a pharmaceutically-acceptable carrier selected from the group consisting of a solvent, a dispersion media, a coating, a stabilizing agent, a diluent, a preservative, an antimicrobial agent, an antifungal agent, an isotonic agent, and an adsorption delaying agent.

Yet another aspect of the disclosure provides a partial coronavirus spike protein comprising a non-glycosylated receptor binding domain (RBD), wherein the non-glycosylated RBD has at least 85% sequence homology with SEQ ID NO: 7.

A further aspect of the disclosure provides a composition comprising: at least one partial coronavirus spike protein comprising a non-glycosylated receptor binding domain (RBD), wherein the non-glycosylated RBD has at least 85% sequence homology with SEQ ID NO: 7; and a pharmaceutically-acceptable carrier selected from the group consisting of a solvent, a dispersion media, a coating, a stabilizing agent, a diluent, a preservative, an antimicrobial agent, an antifungal agent, an isotonic agent, and an adsorption delaying agent.

In some embodiments, the coronavirus spike protein is a SARS-COV-2 spike protein, the at least one partial coronavirus spike protein comprises at least one full-length coronavirus spike protein, the non-glycosylated RBD comprises residues 333-527 of the coronavirus spike protein, and/or the non-glycosylated RBD has at least 90%, at least 93%, at least 95%, at least 97%, or at least 99% sequence homology.

Another aspect of the disclosure provides a nanoparticle comprising at least one protein having at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof.

Yet another aspect of the disclosure provides a nanoparticle comprising at least one partial coronavirus spike protein comprising a receptor binding domain (RBD), wherein the RBD has at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof.

A further aspect of the disclosure provides a nanoparticle comprising at least one non-glycosylated receptor binding domain (RBD) recombinant protein having at least 85% sequence homology with SEQ ID NO: 7.

Another aspect of the disclosure provides a nanoparticle comprising at least one partial coronavirus spike protein comprising a non-glycosylated receptor binding domain (RBD), wherein the non-glycosylated RBD has at least 85% sequence homology with SEQ ID NO: 7.

In some embodiments, the nanoparticle further comprises at least one nanoparticle platform selected from the group consisting of ferritin, dihydrolipoyl acetyltransferase (E2P), Lumazine Synthase (LuS), hepatitis B surface antigen (HBsAg), and human papilloma virus L1 (HPV L1).

Yet another aspect of the disclosure provides a composition comprising a receptor binding domain (RBD) recombinant protein comprising at least one amino acid change selected from the group consisting of Ala 363 changed to a larger amino acid, Ile 468 changed to a polar or charged amino acid, His 519 changed to a more hydrophilic amino acid, Ala 522 changed to Pro, and any combination thereof.

In some embodiments, the large amino acid is selected from the group comprising Tyr, Phe, Trp, Ile, Leu, Met, Val, Lys, Arg, and His. In some embodiments, the large amino acid is selected from the group comprising Tyr, Phe, and Trp. In some embodiments, the polar or charged amino acid is selected from the group consisting of Thr, Ser, Asn, Gln, Glu, Asp, His, Arg, and Lys. In some embodiments, the polar or charged amino acid is selected from the group consisting of Thr, Ser, Asn, Gln, Glu, Asp, Arg, and Lys. In some embodiments, the more hydrophilic amino acid is selected from the group consisting of Asp, Glu, Lys, Arg, Gln, and Asn. In some embodiments, the more hydrophilic amino acid is selected from the group consisting of Asp and Glu. In some embodiments, the at least one amino acid change is selected from the group consisting of A363Y, I468T, H519D, A522P, and any combination thereof.

A further aspect of the disclosure provides a composition comprising at least a partial coronavirus spike protein comprising a receptor binding domain (RBD), wherein the RBD comprises at least one amino acid change selected from the group consisting of Ala 363 to Tyr and homologous substitutions thereof, Ile 468 to Thr and homologous substitutions thereof, His 519 to Asp and homologous substitutions thereof, Ala 522 to Pro, and any combination thereof.

Another aspect of the disclosure provides a composition comprising a non-glycosylated receptor binding domain (RBD) recombinant protein comprising at least one amino acid change selected from the group consisting of Ala 363 to Tyr and homologous substitutions thereof, Ile 468 to Thr and homologous substitutions thereof, His 519 to Asp and homologous substitutions thereof, Ala 522 to Pro, and any combination thereof.

Yet another aspect of the disclosure provides a composition comprising at least a partial coronavirus spike protein comprising a non-glycosylated receptor binding domain (RBD), wherein the non-glycosylated RBD comprises at least one amino acid change selected from the group consisting of Ala 363 to Tyr and homologous substitutions thereof, Ile 468 to Thr and homologous substitutions thereof, His 519 to Asp and homologous substitutions thereof, Ala 522 to Pro, and any combination thereof.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A-D is an exemplary embodiment of an overview of the SPEEDesign pipeline used to create RBD immunogens in accordance with the present disclosure. FIG. 1A shows the SARS-COV-2 spike trimer (green) binds human Ace2 (cyan) to mediate viral entry. This interaction is mediated by the “up” conformation of the RBD (grey), which can also exist in a down conformation (black). FIG. 1B shows the RBD design process retained the Ace2-interaction surface (cyan) and all known SARS neutralizing epitopes (blue). Residues exposed upon isolation of the RBD (green) were heavily designed while all other residues (grey) were designed more conservatively. FIG. 1C shows four computational strategies were used to create 40,000 decoys, each of which has an average of 10 amino acid changes (red) from the native SARS-COV-2 sequence. FIG. 1D shows 28 sequences sampling the top scoring decoys were screened in vitro, identifying 5 lead candidates (stars).

FIG. 2A-D is an exemplary embodiment of higher stability and yield for immunogens than for WT RBD in accordance with the present disclosure. FIG. 2A shows all five immunogens express at higher levels than WT RBD and elute as monomers by size-exclusion chromatography. FIG. 2B shows SDS-PAGE confirms the high purity and yield of RBD immunogens. FIG. 2C shows differential scanning fluorimetry indicates that 4 immunogens have higher thermostability than WT RBD. FIG. 2D shows a summary of Tm and purification yield averages with standard deviations from triplicate purifications.

FIG. 3 is an exemplary embodiment of the sequences of lead immunogens in accordance with the present disclosure.

FIG. 4A-B is an exemplary embodiment of the structural basis for immunogen stabilization in accordance with the present disclosure. FIG. 4A shows sequence alignment of amino acid changes in lead immunogens relative to the native RBD sequence. Four recurring changes are highlighted in pink. FIG. 4B shows the crystal structure of lead immunogen 3 (green) is globally similar to WT RBD (grey; PDB: 7BWJ) despite eight amino acid changes (pink spheres). Insets illustrate key substitutions.

FIG. 5 is an exemplary embodiment of X-ray data collection and refinement statistics in accordance with the present disclosure.

FIG. 6A-C is an exemplary embodiment of neutralizing epitopes unperturbed on RBD immunogens in accordance with the present disclosure. FIG. 6A shows five distinct three-dimensional neutralizing epitopes covering the majority of the protein surface were probed for each immunogen (Ace2: cyan, REGN10933: red, P2B-2F6: orange, S309: green, CR3022: blue).

FIG. 6B shows ELISA probes bind to all five epitopes in the immunogens FIG. 6C shows representative BLI traces used to quantitatively measure the binding of the immunogens to two probes, demonstrating the high integrity of these epitopes. Immunogen concentrations begin at 150 nM and decrease in 2-fold increments.

FIG. 7 is an exemplary embodiment of binding affinities of Ace2 and REG10933 to RBD variants as determined by BLI in accordance with the present disclosure. Binding data were fitted using a 1:1 binding model. The averages and standard deviations of three biological replicates using independently purified immunogens are shown.

FIG. 8A-G is an exemplary embodiment of higher neutralizing antibody titers in mice immunized with immunogens than in WT RBD in accordance with the present disclosure. FIG. 8A shows immunization and blood draw schedule for CD-1 mice. FIG. 8B shows serum ELISA titers against trimeric-FL-spike. Dashed line indicates detection limit of assay and bars represent geometric mean titers (GMT). FIG. 8C shows titers of antibodies blocking Ace2/RBD interaction depicted as described in FIG. 8B. FIG. 8D shows pseudovirus neutralization titers depicted as described in FIG. 8B. Statistical comparisons were made using a Kruskal-Wallis ANOVA followed by Dunn's comparison with WT RBD (*=p<0.05). p-values are 0.29, 0.15, 0.036, and 0.058 for 1, 2, 3, and FL-spike, respectively. FIG. 8E shows purification profiles of FL-spike immunogens containing the modified RBD sequences. Similar peak heights and elution volumes indicating that the RBD mutations do not substantially impact expression and structure. FL-S-1 is the Full-length spike protein ectodomain (with 2P and furin cleavage site mutations) containing an RBD sequence that matches SEQ ID NO: 2 (decoy 28). FL-S-2 is the Full-length spike protein ectodomain (with 2P and furin cleavage site mutations) containing an RBD sequence that matches SEQ ID NO: 3 (decoy 24). FL-S-3 is the Full-length spike protein ectodomain (with 2P and furin cleavage site mutations) containing an RBD sequence that matches SEQ ID NO: 4 (decoy 25). The slightly earlier elution of FL-S-1 may indicate an extended RBD-up conformation. Recombinant FL-spike antigens containing enhancing RBD mutations were expressed in mammalian cell culture and purified by size-exclusions chromatography (SEC). FIG. 8F and FIG. 8G show higher Spike/Ace2 blocking titers for full-length spike incorporated with lead 1 over full-length WT in mice for both the original WA-1 viral variant (FIG. 8F) and the resistant Beta variant (FIG. 8G). Outbred CD-1 mice were immunized twice with 1 μg antigen formulated in the clinically relevant adjuvant AddaSO3. Spike/Ace2-blocking antibody titers were measured 14 days after the second immunization.

FIG. 9A-B is an exemplary embodiment of 1468 and H519 buried in the RBD-down conformation in accordance with the present disclosure. FIG. 9A shows I468 and H519 (pink) are solvent exposed when the RBD (grey) is in the up conformation (PDB 6VSB and 6MOJ) (1, 24). Protomers are shown in green, cyan, and magenta with the RBD of the green protomer in the up conformation. FIG. 9B shows 1468 and H519 (pink) become buried in the closed conformation where they make contacts with the neighboring protomer (cyan) (PDB 6XLU). In some embodiments, mutation of these residues to more hydrophilic residues promotes the RBD-up conformation.

FIG. 10A-C is an exemplary embodiment of deGlyc-SPEEDesign of a non-glycosylated RBD immunogen in accordance with the present disclosure. FIG. 10A shows ELISA of cell-free supernatant from expi293F expression demonstrates that mutation of the N-linked glycosylation site in RBD (T345A) eliminates protein expression. FIG. 10B shows design strategy for a non-glycosylated RBD immunogen in which the epitopes of Ace2 blocking antibodies (red) and/or the Ace2 binding site (blue) are fixed while residues contacting the N-glycan (orange/yellow) are allowed to sample a diverse sequence space. Intermediate residues (grey) are constrained by DMS data. FIG. 10C shows an ELISA expression screen identifies one non-glycosylated immunogen (star) that presents Ace2 and REGN10933 neutralizing epitopes and is expressed at levels comparable to glycosylated immunogens.

FIG. 11A-B is an exemplary embodiment of a homogenous immunogen RBDdGlyc with enhanced expression and thermostability in accordance with the present disclosure. FIG. 11A illustrates that reducing SDS-PAGE of purified WT RBD and RBDdGlyc increased yield and decreased size of the immunogen. FIG. 11B shows DSF melt curves reveal an increased thermostability of RBDdGlyc relative to WT RBD.

FIG. 12A-C is an exemplary embodiment of RBDdGlyc assembly into single-component nanoparticles presenting neutralizing epitopes in accordance with the present disclosure. FIG. 12A shows SDS-PAGE of cell-free supernatant from expi293F expression demonstrates that single-component RBDdGlyc nanoparticles show high expression levels, while glycosylated WT RBD nanoparticles do not. FIG. 12B shows EM images of purified RBDdGlyc nanoparticles demonstrate efficient and homogenous assembly. FIG. 12C shows ELISAs on purified RBDdGlyc nanoparticles with neutralizing antibodies demonstrate the accessibility of neutralizing epitopes.

FIG. 13A-B is an exemplary embodiment of RBDdGlyc nanoparticles eliciting higher titers of blocking antibodies than monomeric RBD and comparable titers to FL-S-2P in accordance with the present disclosure. Outbred CD-1 mice were immunized twice with 1 μg antigen formulated in Addavax. FIG. 13A shows spike/Ace2-blocking antibody titers were measured 14 days after the second immunization against the original WA-1 viral variant. FIG. 13B shows spike/Ace2-blocking antibody titers were measured 14 days after the second immunization against the resistant Beta variant.

In the present application, SEQ ID NO: 1 is an amino acid sequence of SARS-COV-2 Spike Protein Native RBD; SEQ ID NO: 2 is an amino acid sequence of Recombinant RBD Immunogen, Lead 1, Decoy 28; SEQ ID NO: 3 is an amino acid sequence of Recombinant RBD Immunogen, Lead 2, Decoy 24; SEQ ID NO: 4 is an amino acid sequence of Recombinant RBD Immunogen, Lead 3, Decoy 25; SEQ ID NO: 5 is an amino acid sequence of Recombinant RBD Immunogen, Lead 4, Decoy 26; SEQ ID NO: 6 is an amino acid sequence of Recombinant RBD Immunogen, Lead 5, Decoy 21; SEQ ID NO: 7 is an amino acid sequence of Non-glycosylated RBD.

### DETAILED DESCRIPTION

The following detailed description and examples set forth preferred materials and procedures used in accordance with the present disclosure. It is to be understood, however, that this description and these examples are provided by way of illustration only, and nothing therein shall be deemed to be a limitation upon the overall scope of the present disclosure.

Novel compositions of matter having better neutralizing responses than current spike-based immunogens for COVID-19 vaccines and new Stabilizer for Protein Expression and Epitope Design (SPEEDesign) computational design pipeline for in vitro screening

New compositions of matter are disclosed herein comprising novel protein sequences that can be used as coronavirus vaccine candidates. These sequences are not found in nature, but they are derived from the receptor binding domain (RBD) of the spike protein, a leading target for vaccine development. A computational pipeline was created and implemented to design improved stabilized antigens that present the protective epitopes on the RBD. These designed antigen sequences contain engineered amino acid changes that increase protein yield, increase protein stability, and result in an improved immune response upon immunization of animals.

Specifically, the designs disclosed herein can be produced approximately 7 times more efficiently than the native sequence, facilitating vaccine manufacturing on a global scale. The disclosed designs also have up to 10° C. higher thermal stability than the native sequence, suggesting enhanced stability during storage and when in the body. Finally, immunization of animals with the disclosed antigens produces up to 10-fold higher levels of blocking antibodies than the native sequence and 30-fold higher levels of pseudoviral neutralizing antibodies. The disclosed engineered RBD antigens have enhanced biophysical and immunological characteristics that translate into significant improvements in pre-clinical animal models and they will be a central component of potent coronavirus vaccines.

As described herein, novel RBD immunogens were created with enhanced characteristics for production, storage, distribution, and vaccine efficacy. A new computational design and in vitro screening pipeline (i.e., SPEEDesign) was implemented to identify 5 lead candidates for development, each with approximately 9 amino acid changes relative to the native RBD sequence. These lead candidates have increased production yields and thermostability, and they retain structured neutralizing epitopes. Immunization of mice with monomeric immunogens results in higher levels of neutralizing antibodies than native RBD, and levels similar, or greater than, trimeric FL-spike harboring stabilizing mutations. These results are shown herein below in Examples 1-5.

The RBD antigens disclosed herein are more immunogenic, more stable, and more easily produced than antigens currently in use. Current coronavirus vaccines use the naturally occurring RBD sequence in isolation, or in the context of the full-length spike protein. These designed antigens are more immunogenic than the natural sequence in animals, suggesting that they would elicit stronger, more consistent, and more durable protection in humans. The antigens disclosed herein are also more efficiently expressed, suggesting decreased cost of production and/or dose sparing capabilities. Finally, the increased thermostability of these antigens indicate advantages in storage and distribution.

The antigens disclosed herein have the potential to enter human clinical trials and be approved as a COVID-19 vaccine under accelerated development timelines used for other COVID-19 vaccines. This usage includes recombinant protein, mRNA, self-amplifying RNA, DNA, viral-vectored, inactivated virus, or any other vaccine platform that presents coronavirus proteins to the human body.

In some embodiments, the proteins described herein can be used to test for the presence of neutralizing antibodies against SARS-COV-2 in humans or animals. In some embodiments, this protein can be used as a therapeutic that competes with the RBD of infectious virus for receptor interaction. In some embodiments, the proteins described herein can be used to isolate neutralizing antibodies from patients or phage-display libraries. In some embodiments, the proteins described herein can be used as a stabilized scaffold for the presentation of epitopes from homologous proteins. In some embodiments, the proteins described herein can be modified to form a nanoparticle vaccine that increase its immunogenicity.

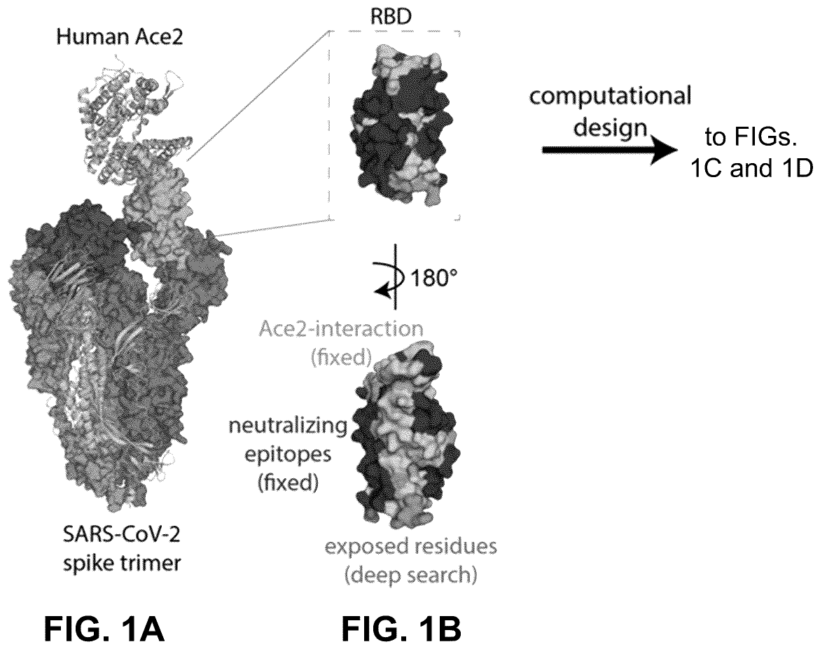

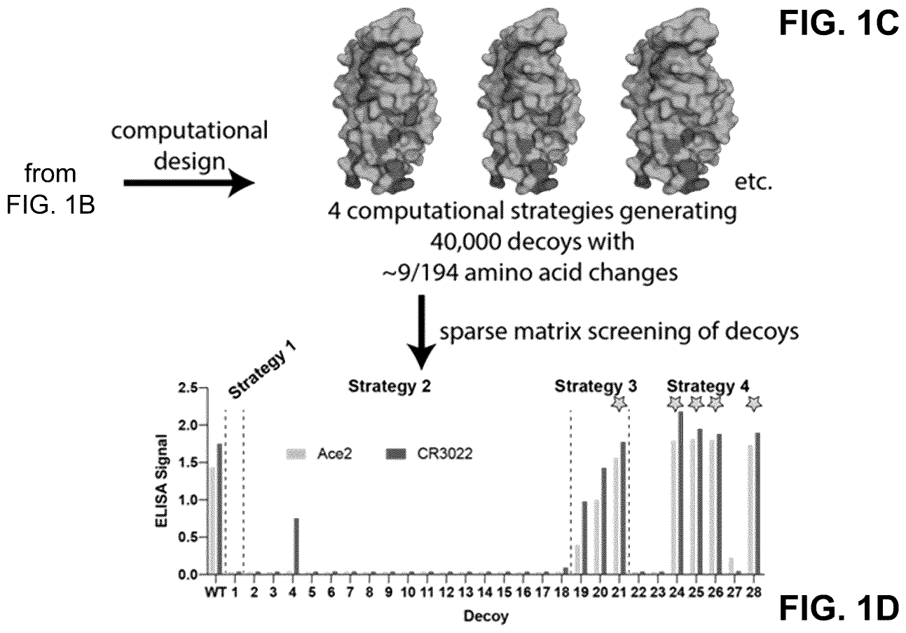

The receptor binding domain (RBD) of the spike protein is the target of the most potent SARS-COV2 neutralizing antibodies (FIGS. 1A and B). In fact, the RBD alone is a vaccine candidate that elicits potent neutralizing antibodies and protective immunity in humans and animals. However, these potent neutralizing antibodies are extremely rare in convalescent patients relative to non-neutralizing antibodies that bind epitopes elsewhere on the spike protein. This phenomenon is likely due to structural instability in neutralizing epitopes of the RBD and the ability of the RBD to adopt a “down” conformation that obscures neutralizing epitopes in the FL-spike trimer (FIG. 1A). As disclosed herein, the RBD transiently samples an “up” conformation compatible with Ace2 binding, which exposes neutralizing epitopes, including some that may cross-protect against other coronaviruses. Exposure of the RBD, either as a stand-alone vaccine or in the RBD-up conformation of the spike trimer, and stabilization of the RBD structure is expected to increase the immune response to neutralizing epitopes. As disclosed herein, the novel SPEEDesign pipeline identifies stabilizing amino acid changes to the receptor binding domain of SARS-COV-2 spike, improving biophysical characteristics and vaccine efficacy in mice. Detailed methods of this novel design pipeline are demonstrated herein below in Example 6.

### Design of a Non-Glycosylated RBD Enables a Single-Component SARS-COV-2 Nanoparticle Vaccine

As described above, the spike protein RBD is the target of the most potent neutralizing antibodies against SARS-COV-2. RBD residues 333-527 contain an N-linked glycosylation site at position 343 where large heterogeneous sugars are added post-translationally. The size and composition of these glycans depends on many factors, including cell type used for expression. These variable factors mean that glycoprotein characteristics can vary between expression platforms and between batches. Variability in glycan composition can have a major impact on the immunogenicity, stability, solubility, and efficacy of a vaccine, leading to challenges associated with the production of homogenous reproducible glycoprotein vaccines. Removing the N-linked glycosylation site in the RBD has the potential to improve homogeneity, reproducibility, and efficacy of a SARS-COV-2 vaccine candidate.

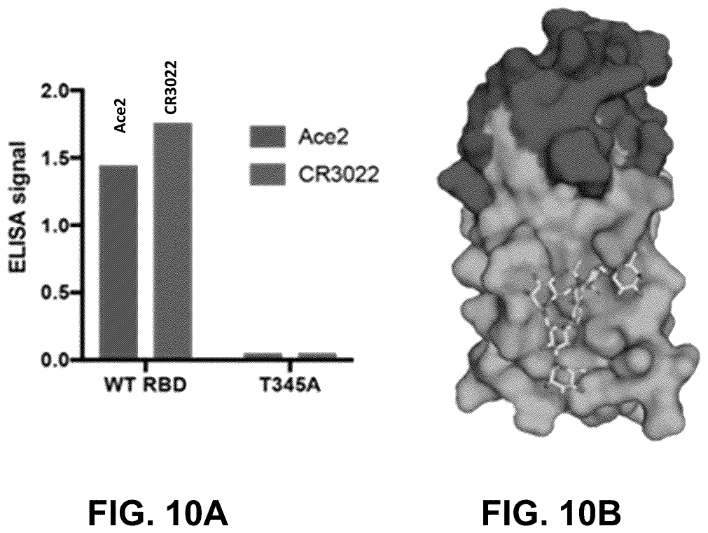

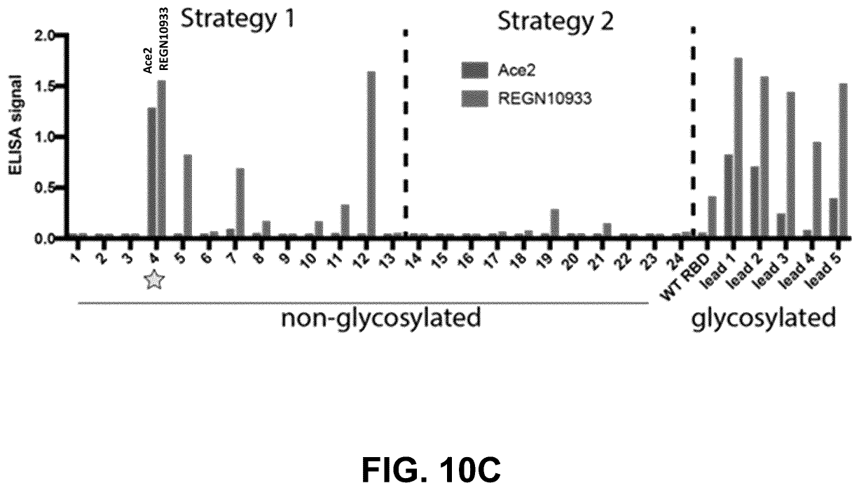

The simplest manner of removing N-linked glycosylation is to mutate the NxS/T recognition motif. However, mutation of this motif prevents protein expression. Changing threonine 345 to alanine eliminates this motif and significantly reduces expression in expi293F cells (FIG. 10A). This result is consistent with deep mutational scanning (DMS) data that shows a significant decrease in yeast expression upon mutation of either residue that comprises the NxS/T motif. The dependence of protein expression on this motif suggests that the glycan plays a stabilizing role structurally.

As disclosed herein, the decrease in protein expression upon removal of the NxS/T motif is caused by a decrease in thermodynamic stability of the RBD, and additional amino acid changes to the residues surrounding the glycan compensate for the lost glycan to restore protein expression. These results are shown herein below in Examples 7-9.

An “immunogenic or immunological composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host. It should be appreciated that all scientific and technological terms used herein have the same meaning as commonly understood by those of ordinary skill in the art.

Additionally, the composition can include one or more pharmaceutical-acceptable carriers. As used herein, “a pharmaceutical-acceptable carrier” includes any and all solvents, dispersion media, coatings, stabilizing agents, diluents, preservatives, antibacterial and antifungal agents, isotonic agents, adsorption delaying agents, and the like. Optionally, some embodiments include the addition of a protectant. A protectant as used herein refers to an anti-microbiological active agent. In particular adding a protectant is most preferred for the preparation of a multi-dose composition. Those anti-microbiological active agents are added in concentrations effective to prevent the composition of interest from any microbiological contamination or for inhibition of any microbiological growth within the composition of interest. The compositions of the present disclosure can also comprise the addition of any stabilizing agent, such as for example saccharides, trehalose, mannitol, saccharose and the like, to increase and/or maintain product shelf-life and/or to enhance stability. In preferred forms, the composition may also include additional components known to those of skill in the art (see also Remington's Pharmaceutical Sciences, 1990, 18th ed. Mack Publ., Easton). Those of skill in the art will understand that the composition herein may incorporate known injectable, physiologically acceptable, sterile solutions. For preparing a ready-to-use solution for parenteral injection or infusion, aqueous isotonic solutions, such as e.g. saline or corresponding plasma protein solutions are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others. Suitable adjuvants are those additional components known to those of skill in the art. When administered as a liquid, a vaccine composition of the present disclosure may be prepared in the form of an aqueous solution, syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like. According to a further aspect, the immunogenic composition of the present disclosure further comprises a pharmaceutical acceptable salt, preferably a phosphate salt in physiologically acceptable concentrations. Preferably, the pH of said immunogenic composition is adjusted to a physiological pH, meaning between about 6.5 and 7.5. The immunogenic compositions described herein can further include one or more other immunomodulatory agents such as, e.g., interleukins, interferons, or other cytokines. The immunogenic compositions can also include antibiotics or anti-microbiological active agents. It will be found that the immunogenic compositions comprising recombinant RBD protein(s) as provided herewith are very effective in reducing the severity of or incidence of clinical signs associated with SARS-COV-2 infections up to and including the prevention of such signs.

The present disclosure relates to compositions of matter comprising at least one recombinant RBD protein, at least one nucleotide encoding a respective RBD protein, at least one partial coronavirus spike protein comprising an RBD protein, and/or a nanoparticle comprising an RBD protein. In some embodiments, this composition of matter also comprises an agent suitable for the inactivation of viral vectors. Such products are useful as immunogenic compositions that induce an immune response and, more preferably, confers protective immunity against the clinical signs of SARS-COV-2 infection. The composition generally comprises at least one protein having at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof, as the antigenic component of the composition. It is understood by those of skill in the art that each sequence selected from the group consisting of SEQ ID NO: 2, SEQ ID NO: 3, SEQ ID NO: 4, SEQ ID NO: 5, SEQ ID NO: 6 could vary by as much as 5% in sequence homology and still retain the antigenic characteristics that render it useful in immunogenic compositions. In some embodiments, compositions of the present disclosure comprise at least one non-glycosylated RBD protein, at least one nucleotide encoding a non-glycosylated RBD protein, at least a partial coronavirus spike protein comprising a non-glycosylated RBD protein, and/or a nanoparticle comprising a non-glycosylated RBD protein having at least 85% sequence homology with SEQ ID NO: 7 as the antigenic component of the composition. Further, it is understood by those of skill in the art that a protein having SEQ ID NO: 7 could vary by as much as 15% in sequence homology and still retain the antigenic characteristics that render it useful in immunogenic compositions.

The compositions as described herein preferably may be applied intramuscularly, intravenously, or intranasally. The amount of a vaccine that is effective depends on the ingredients of the vaccine and the schedule of administration. A vaccine composition of the present disclosure can be administered in a single dose or in repeated doses, with a single dose being preferred. Depending on the desired duration and effectiveness of the treatment, repeated doses of immunogenic compositions according to the disclosure may be administered once or several times, also intermittently, for instance on a daily, weekly, or monthly basis for several days, weeks or months, and in different dosages.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12 (1): 387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, MD 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

A “conservative substitution” or a “homologous substitution” refers to the substitution of an amino acid residue or nucleotide with another amino acid residue or nucleotide having similar characteristics or properties including size, hydrophobicity, etc., such that the overall functionality does not change significantly.

This written description uses examples to describe the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLE 1

This example demonstrates SPEEDesign of novel RBD immunogens.

A novel computational design and in vitro screening pipeline was used to develop improved RBD vaccine candidates. Stabilizer for Protein Expression and Epitope Design (SPEEDesign) is a pipeline that retains neutralizing epitopes while stabilizing protein domains and removing non-neutralizing epitopes. In the case of the RBD, the Ace2 interface and all known neutralizing epitopes were unchanged (FIG. 1B). Residues that are exposed in the RBD “up” conformation, or that are exposed upon extraction of the RBD from the FL-spike protein, were thoroughly searched during the design process to identify amino acid changes that would stabilize an accessible RBD. All remaining residues were allowed to sample a limited sequence space defined by energetic and evolutionary restraints.

Four different computational ROSETTA design strategies were used to identify amino acid changes that would stabilize the RBD (FIG. 1C). Ten thousand decoys were produced for each computational strategy, each of which had approximately 9 amino acid changes from the native RBD sequence. Twenty-eight decoys were selected using a clustering algorithm designed to broadly sample the 40,000 computational decoys (FIG. 1D). These decoys are distributed between strategies according to the sequence diversity produced by each strategy. For example, more decoys were selected from strategy 2 than strategy 1 because strategy 2 samples a much larger sequence space.

The 28 representative sequences were screened in vitro for expression and presentation of neutralizing epitopes (FIG. 1D). ELISA probes were used that recognize the Ace2 interface and CR3022 antibody epitope to identify five lead immunogens that were expressed at levels comparable to the native wild-type (WT) sequence and that present the key neutralizing epitopes that overlap with the Ace2 binding site or are recognized by CR3022. Interestingly, all lead candidates derived from strategies 3 and 4, which sample a moderate sequence space, rather than the highly restricted strategy 1 or highly divergent strategy 2. Additionally, the immunogen with the best ROSETTA score from these strategies (decoys 19 and 22) were not successful, indicating that ROSETTA score alone is not a suitable predictor for a successful design. The clustering and screening strategies unique to the SPEEDesign pipeline are a critical advancement that allows the identification of improved immunogens among the many decoys produced during ROSETTA design.

### EXAMPLE 2

This example demonstrates the increased expression and stability of designed RBD immunogens.

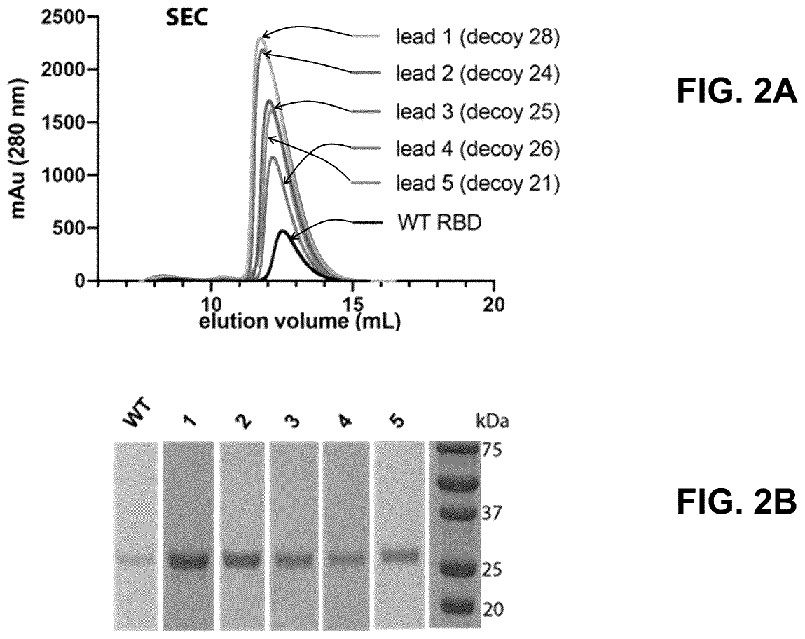

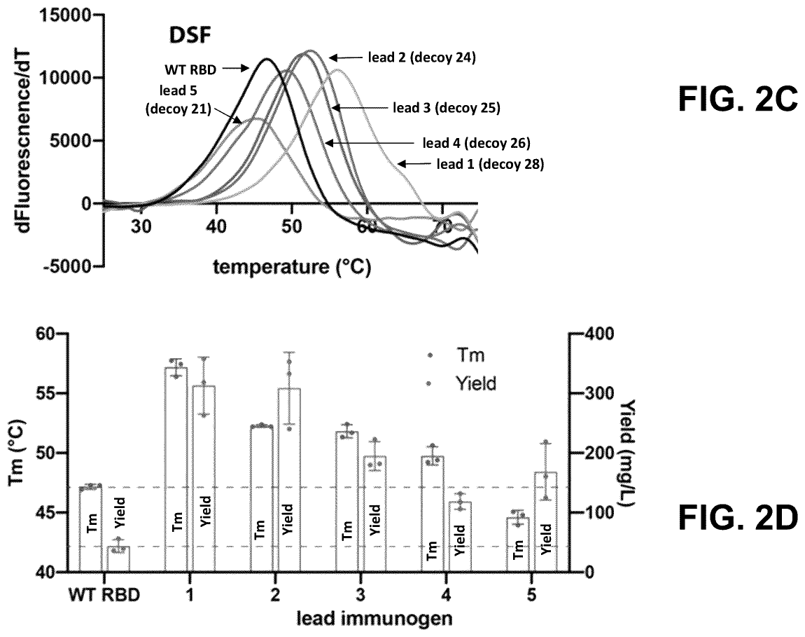

Five lead candidate immunogens (leads 1-5, corresponding to decoys 28, 24, 25, 26, and 21, respectively) were expressed and purified to determine their biophysical characteristics. All immunogens are well-folded monomeric proteins that can be highly purified with yields much greater than WT RBD (FIGS. 2A, B, and D). Three immunogens (1, 2, and 3) can be purified with final yields ≥200 mg/L, or more than 4-fold higher than WT RBD (FIG. 2D). Furthermore, these yields are approximately 6-fold higher than the yields reported for an optimized FL-spike ectodomain known as “hexapro” and 200-fold higher than the well-established 2P prefusion-stabilized FL-spike ectodomain, or expressed as a per mole or per epitope equivalent these improvements are 35 and 1,200 fold higher. These yields were achieved without optimization of the expression and purification system, suggesting that process development is capable of reaching production levels sufficient for the development of a highly cost-effective vaccine.

The thermostability of four lead immunogens was also higher than WT RBD (FIGS. 2C and D). One immunogen (1) has a melting temperature (Tm) of 57° C., or 10° C. higher than WT RBD, and two additional immunogens (2 and 3) have greater than 5° C. increases in Tm. Expression yield roughly correlates with Tm, and immunogens 1, 2, and 3 have substantially higher yields and stability than WT RBD. Increased thermostability likely contributes to the increase in immunogen yield, may increase the half-life of the antigen in the body, and may improve stability during storage, transportation, and administration of a vaccine, alleviating cold-chain requirements.

### EXAMPLE 3

This example demonstrates the structural basis for enhanced immunogen stability.

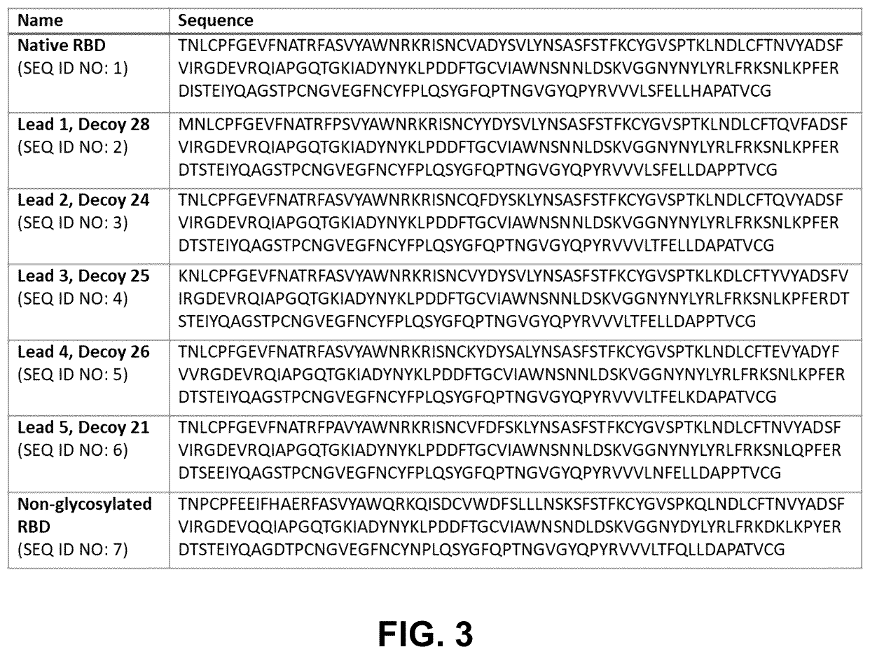

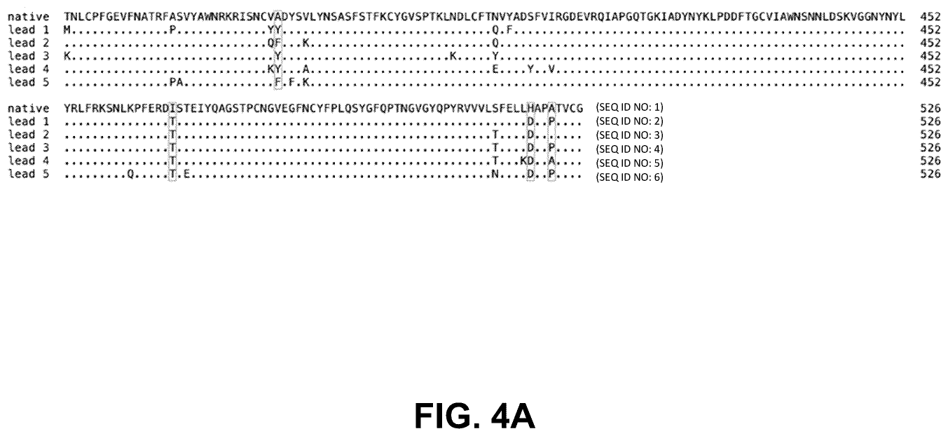

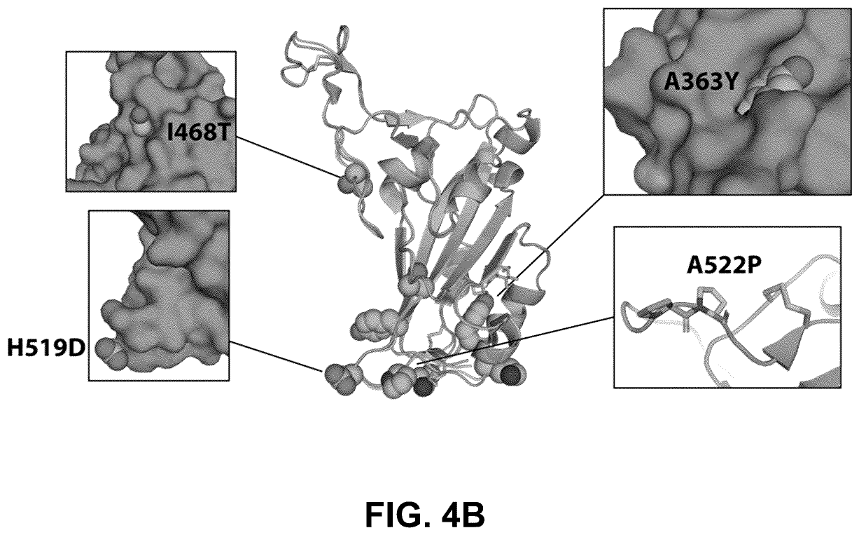

The five lead immunogens have a distinct pattern of amino acid changes that provide a mechanistic basis for their enhanced biophysical characteristics. There are nine positions at which multiple lead candidates have non-conservative amino acid substitutions relative to the native protein sequence (FIG. 3 and FIG. 4A). At four of these positions, the immunogens share similar amino acid identities: 1) Alanine 363 is changed to a large hydrophobic residue in all five lead immunogens, 2) I468T in all five leads, 3) H519D in all five leads, 4) A522P in 3/5 leads. In some embodiments, a composition comprises an RBD recombinant protein or at least a partial coronavirus spike protein comprising the RBD. In these embodiments, the RBD comprises at least one amino acid change selected from the group consisting of Ala 363 to Tyr and homologous substitutions thereof, Ile 468 to Thr and homologous substitutions thereof, His 519 to Asp and homologous substitutions thereof, Ala 522 to Pro, and any combination thereof.

Accordingly, in some embodiments, a composition comprises an RBD recombinant protein or at least a partial coronavirus spike protein comprising the RBD. In these embodiments, the RBD comprises at least one amino acid change selected from the group consisting of Ala 363 changed to a larger amino acid, Ile 468 changed to a polar or charged amino acid, His 519 changed to a more hydrophilic amino acid, Ala 522 changed to Pro, and any combination thereof. In some embodiments, the large amino acid is selected from the group comprising Tyr, Phe, Trp, Ile, Leu, Met, Val, Lys, Arg, and His, or is selected from the group comprising Tyr, Phe, and Trp. In some embodiments, the polar or charged amino acid is selected from the group consisting of Thr, Ser, Asn, Gln, Glu, Asp, His, Arg, and Lys, or is selected from the group consisting of Thr, Ser, Asn, Gln, Glu, Asp, Arg, and Lys. In some embodiments, the more hydrophilic amino acid is selected from the group consisting of Asp, Glu, Lys, Arg, Gln, and Asn, or is selected from the group consisting of Asp and Glu. In some embodiments, the at least one amino acid change is selected from the group consisting of A363Y, I468T, H519D, A522P, and any combination thereof.

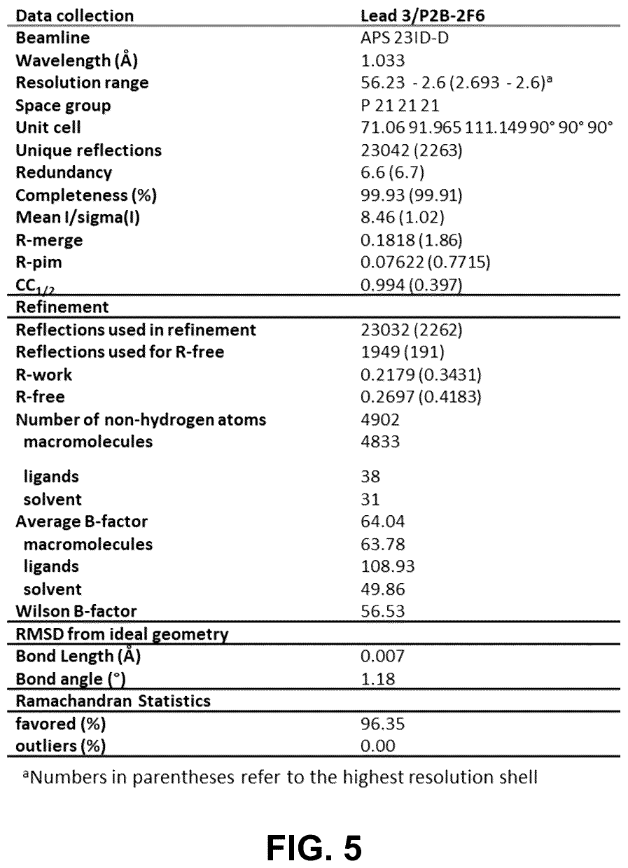

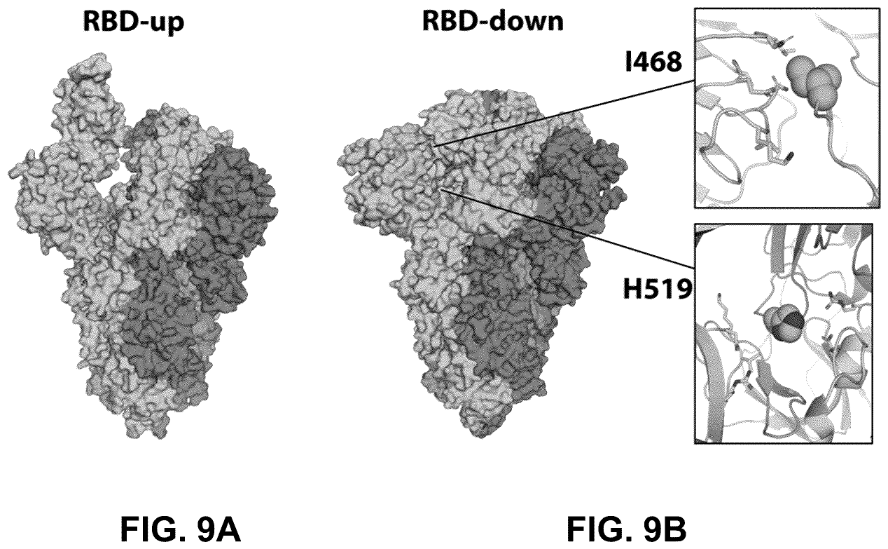

To determine how these amino acid changes improve the stability of the immunogens, the crystal structure of lead immunogen 3 was solved in complex with the neutralizing Fab P2B-2F6. The overall structure of lead 3 is very similar to the native RBD with a Ca RMSD of only 0.476 Å across all residues, indicating that the amino acid changes do not alter the overall shape, secondary structure, or tertiary structure of the RBD (FIG. 4B and FIG. 5). The enhanced biophysical characteristics are driven by local structural changes around substituted side-chains. For example, A363Y is a space-filling substitution that likely stabilizes the protein fold and proximal di-sulfide bonds. Ile468 and His519 are buried in the RBD-down conformation and become exposed in the RBD-up conformation (FIG. 9A-B). Thus, the I468T and H519D substitutions increase the hydrophilicity of solvent-exposed residues, likely improving the solubility of RBD immunogens and potentially promoting the RBD-up conformation in the context of the FL-spike. Finally, A522P creates a tandem proline sequence that likely promotes a sharp kink in the backbone adjacent to a disulfide bond. Accordingly, in some embodiments, a composition comprises a non-glycosylated RBD recombinant protein or at least a partial coronavirus spike protein comprising a non-glycosylated RBD. In these embodiments, the non-glycosylated RBD comprises at least one amino acid change selected from the group consisting of Ala 363 to Tyr and homologous substitutions thereof, Ile 468 to Thr and homologous substitutions thereof, His 519 to Asp and homologous substitutions thereof, Ala 522 to Pro, and any combination thereof. Thus, the stabilizing mutations promote the stability of the immunogens through diverse structural and energetic mechanisms that do not impact the global antigen structure.

### EXAMPLE 4

This example demonstrates that neutralizing epitopes are unperturbed on designed RBD immunogens.

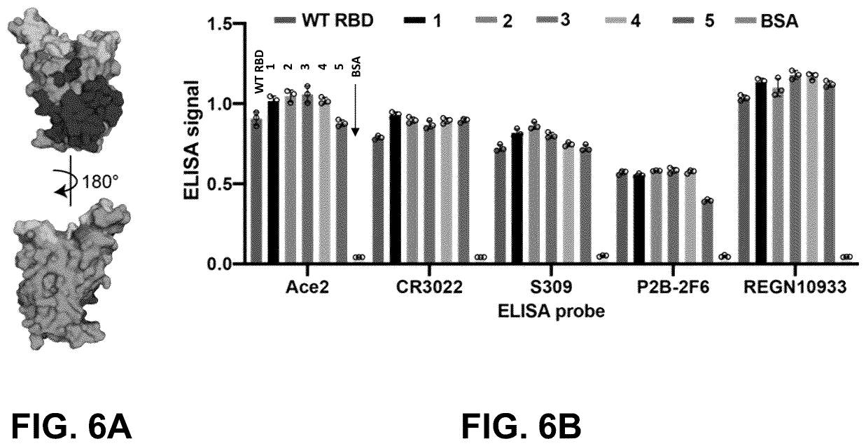

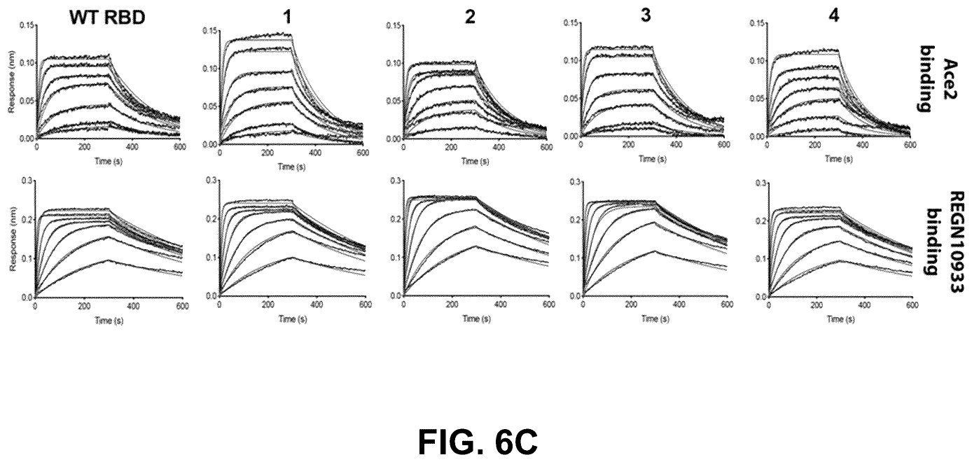

The Ace2 receptor binding site and several key three-dimensional antibody epitopes were probed to establish that the stabilizing mutations did not disrupt key interacting residues or neutralizing epitopes in the immunogens (FIG. 6A-C). Ace2 binds to the end of the RBD and the mAbs CR3022 and S309 bind to opposing sides (FIG. 6A). The Ace2 binding site and the two epitopes are non-overlapping and therefore report the structural integrity of almost all neutralizing surfaces retained during the design process. Ace2 blocking antibodies are the most potent neutralizing antibodies, therefore these epitopes were further investigated with the REGN10933 and P2B-2F6 mAbs. All immunogens bind these probes at least as well as WT, with the exception of lead 5 showing a slight decrease in binding to P2B-2F6, suggesting that the stabilizing changes made to the immunogens do not perturb nearby surfaces (FIG. 6B).

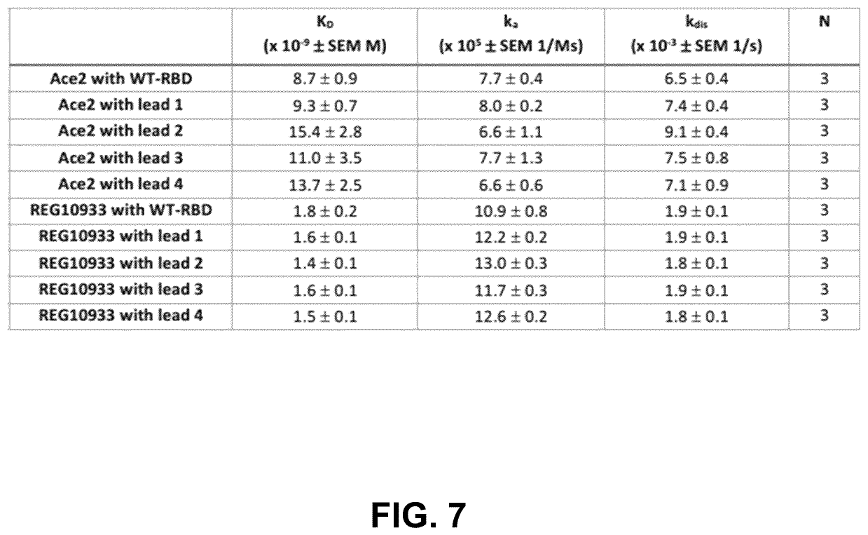

Biolayer interferometry (BLI) was also used to measure the integrity of the Ace2 binding site and REGN10933 epitope in a more quantitative fashion (FIG. 6C). Consistent with the ELISA results, leads 1˜4 bind both probes with biophysical parameters very similar to WT RBD (FIG. 7). While these probes are only a small fraction of the antibodies reported to bind SARS-COV-2, they represent all four classes of epitopes recognized by known RBD antibodies. Therefore, neutralizing epitopes are unperturbed on RBD immunogens 1˜4 and an immune response to an immunogen is expected to recognize the native RBD protein and SARS-COV-2 virus.

### EXAMPLE 5

This example demonstrates that vaccination with designed RBD immunogens elicits an improved protective antibody response.

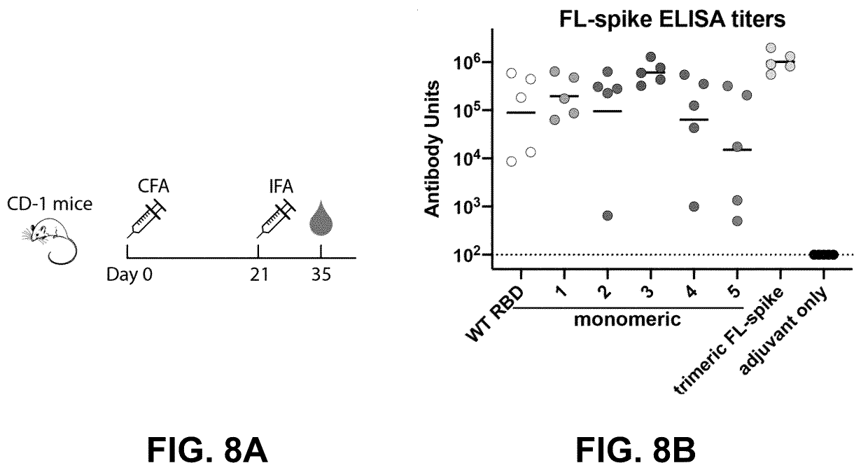

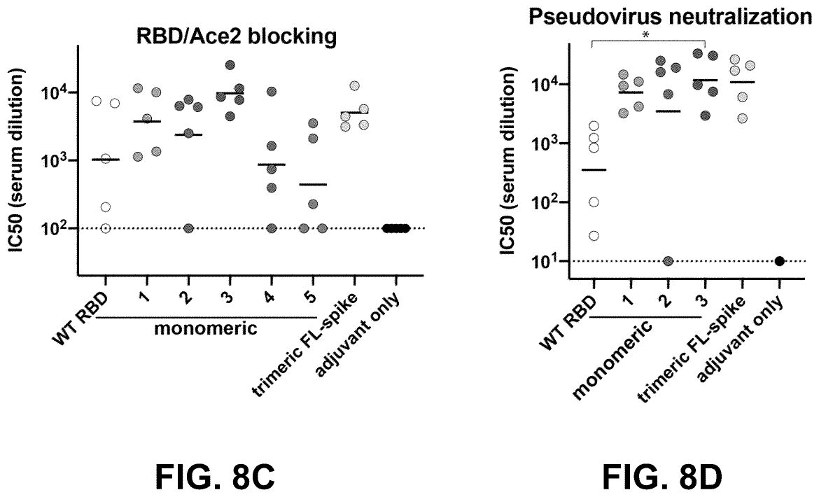

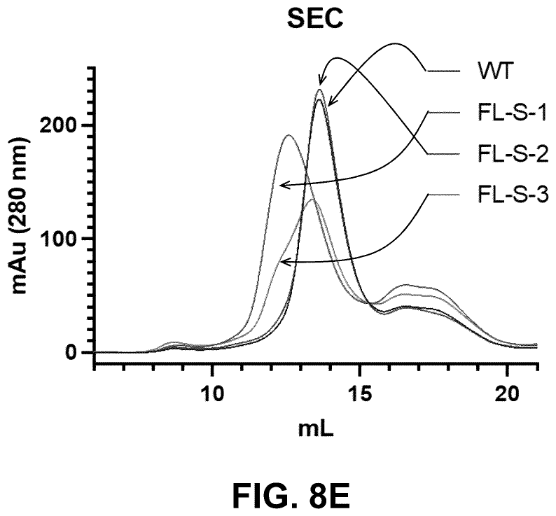

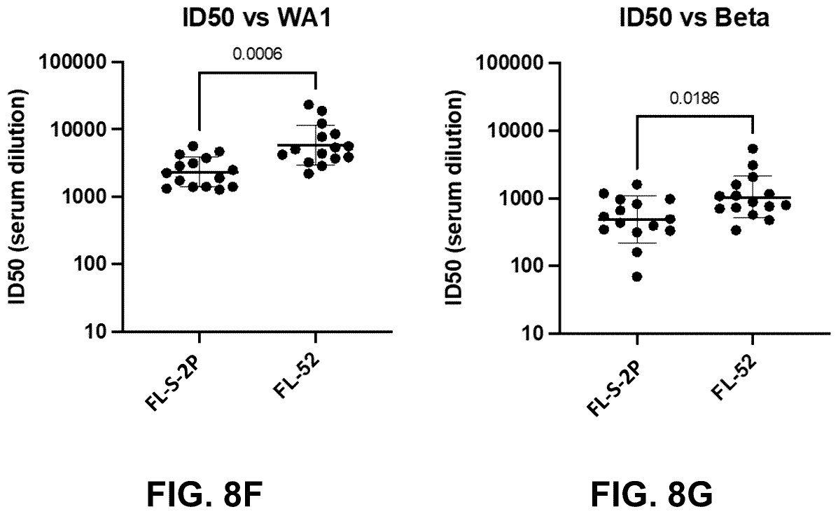

Mice were immunized with the five lead immunogens as monomers to determine if the amino acid changes improve the neutralizing antibody response (FIG. 8A-D). For comparison, mice were immunized with monomeric WT RBD or the trimeric 2P prefusion-stabilized spike ectodomain (trimeric-FL-spike). CD-1 outbred mice were used to mimic the genetic diversity found in the human population more closely than inbred mice. All immunogens generate antibodies that recognize trimeric-FL-spike and 1, 2, and 3 generate geometric mean titers (GMT) greater than WT RBD, consistent with their enhanced biophysical characteristics (FIG. 8B). Trimeric-FL-spike elicits slightly higher levels of antibodies, many of which likely target non-neutralizing epitopes outside the RBD. Inhibition of RBD/Ace2 binding was therefore measured to evaluate the titers of functional antibodies elicited by each antigen (FIG. 8C). Again, 1, 2, and 3 outperform WT RBD, generating Ace2-blocking GMTs up to 10-fold higher. Neutralizing titers in a pseudoviral neutralization assay were also measured and again it was found that 1, 2, and 3 outperform WT RBD (FIG. 8D). Immunogen 3 elicits neutralizing titers significantly greater than WT RBD with a GMT more than 30-fold higher than WT, an even greater enhancement than the ˜10-fold increase provided by the 2P stabilizing mutation. This difference is equivalent to lengthening the lifetime of neutralizing antibodies by approximately five half-lives of decay, suggesting that this immunogen confers a much longer duration of antibody-mediated protection than WT RBD. Additionally, this 30-fold enhancement is greater than the ˜6-fold reduction in neutralizing potential against new SARS-CoV-2 variants, such as B.1.351, suggesting that these immunogens increase the breadth of protection, in addition to the duration. The neutralizing GMT elicited by 3 is also slightly higher than trimeric-FL-spike despite the fact that the designed immunogen is monomeric while the trimeric-FL-spike benefits from the multivalent display of epitopes.

A full-length SARS-COV-2 spike antigen was developed containing the novel enhancing mutations to the RBD described herein. Immunogenicity data in mice demonstrated that this developed full-length antigen is superior to the unmodified full-length antigen. Improvement of RBD antigens by incorporation of the novel amino acid changes of the present disclosure also improved full-length spike antigens. As full-length spike antigens are used in all currently-authorized SARS-COV-2 vaccines, the data of the present disclosure is particularly relevant to the development of improved next-generation SARS-COV-2 vaccines.

Recombinant FL-spike antigens were produced with enhancing mutations to the RBD (FIG. 8E). These experiments demonstrate that the mutations do not disrupt expression or trimerization of the FL-spike protein. Furthermore, the FL-spike immunogen FL-S-1 (SEQ ID NO: 2) elutes slightly earlier from a size exclusion column than the unmodified FL-spike protein. This elution volume is consistent with the extended RBD-up conformation in FIG. 9A. Incorporation of lead 1 into full length spike (FL-S-1) elicited significantly higher titers of Spike/Ace2 blocking titers than FL-S-2P in mice (FIG. 8F-G). This enhancement is present against both the original WA-1 viral variant, as well as the resistant Beta variant.

### EXAMPLE 6

This example demonstrates the materials and methods of SPEEDesign.

At least one of the goals of the Stabilizer for Protein Expression and Epitope Design (SPEEDesign) disclosed herein is to focus the immune response to conformational neutralizing epitopes from an antigen with neutralizing, non-neutralizing and immunodominant epitopes while stabilizing the domain. Within the context of SPEEDesign, four design approaches are contemplated to improve vaccine efficacy. Neutralizing antibody titers can be improved by: (1) focusing the immune response to neutralizing epitopes, (2) eliminating immunogenicity of non-neutralizing epitopes, (3) stabilizing the antigen to lengthen half-life and improve immunogenicity, and (4) promoting transient states. In exemplary embodiments, the core is stabilized by evolutionarily allowed residues from multiple sequence alignments, neutralizing epitope residues remained fixed, exposed residues not under evolutionary constraints are allowed to vary extensively, non-neutralizing/immunodominant epitopes are allowed to mutate. Result candidates are fed into Rosetta design procedure with multiple protocols and clustering analysis samples the most diverse representative designs.

### SPEEDesign Residue Definition

Each amino acid in the target antigen is categorized as fixed, intermediate, or deep search, defining the depth of the computational search at that position. RBD residues that form the interface with Ace2 or neutralizing antibodies were defined as fixed. This design process was performed prior to the isolation of SARS-COV-2 specific antibodies, so epitopes from SARS-1 neutralizing antibodies (CR3022, 80R, m396, F26G19, S230, and VHH-72) were used as a proxy. These epitopes accurately predicted and cover the residues targeted by the SARS-CoV2 antibodies identified after the design process was initiated and therefore retain the SARS-Cov2 neutralizing epitopes as fixed despite not having this information available during design initiation. The residues that comprise these epitopes were defined as those that have a >1 Å change in solvent accessible surface area upon complex formation, and calculations were performed in PyMOL.

Those residues that are exposed in the RBD-up conformation or upon extraction of the RBD from the FL-spike protein were defined as deep search. Residues exposed upon domain extraction from a larger protein require unique handling during the design processes. These residues are buried or interacting with other residues in the larger protein, and they become fully solvent exposed once the domain is extracted. This dramatic change in chemical environment is accommodated by allowing deep search residues to vary greatly during the design process. Since these residues are not exposed in homologous proteins, conservation or evolutionary-based design principles are unlikely to prove sufficient to redesign these new non-natural surfaces. In some embodiments, these residues were therefore classified for deep search during design where all amino acids except cysteine are allowed. In other embodiments, all amino acids are allowed for deep search.

All other residues were defined as intermediate. These residues are allowed to vary to a limited extent that is driven by conservation and evolutionary analysis of similar protein sequences to identify potential amino acid changes.

### SPEEDesign ROSETTA Strategies

All ROSETTA strategies leave fixed residues unchanged to preserve neutralizing epitopes, and each strategy differs in the amino acid changes allowed for the intermediate and deep search categories of residues. In strategy 1, intermediate residues were unchanged and all amino acids except cysteine were allowed at deep search positions. In strategy 2, all amino acids were allowed at deep search positions, and intermediate positions were allowed to sample amino acids found in proteins with similar sequences (evolutionary constraints). Strategy 2 samples a very large sequence space, which is constrained in strategy 3 by disallowing amino acid changes that are energetically unfavorable when made individually (energetic constraints), an approach adapted from the PROSS protocol. Strategy 4 places evolutionary and energetic constraints on the intermediate residues but allows the deep search residues to sample all amino acids.

### SPEEDesign Clustering

For each computational strategy, decoys with scores in the 95th percentile were clustered by sequence similarity and the top scoring decoy form each cluster was selected as a representative sequence. In some embodiments, high-scoring decoy sequences are clustered based on sequence similarity, wherein clustering high-scoring decoy sequences comprises clustering decoy sequences with scores in a 90th percentile, a 95th percentile, a 96th percentile, a 97th percentile, a 98th percentile, or a 99th percentile based on sequence similarity. Those of skill in the art will recognize that any suitable clustering method and/or program may be used for decoy clustering as disclosed herein (e.g., CD-HIT, phylogenetic tree generation followed by internal node sequence screening, etc.). The number of clusters was selected based on the sequence diversity produced in each computational strategy. For example, strategy 1 samples a limited sequence space, while strategy 2 samples a very large sequence space. Therefore, more clusters were created to sample strategy 2 than strategy 1.

### SPEEDesign In Vitro Screening

Synthetic DNA coding for secreted RBD immunogens was cloned (GenScript) into a customized pHL-see expression plasmid. pHL-see was a gift from Edith Yvonne Jones (Addgene plasmid #99845; http://n2t.net/addgene: 99845; RRID: Addgene_99845). Plasmid was transfected into human expi293F cells and grown in a 96-well plate according to manufacturer instructions (ThermoFisher Scientific). Cell-free supernatant was harvested after 5 days of expression.

Cell-free supernatant was diluted in PBST+2% BSA and added to Ni-NTA HisSorb Plates (Qiagen) to capture His-tagged immunogens. After incubation for 1 hour at room temperature, plates were washed three times with PBST. Neutralizing epitopes were probed using an Ace2-Fc (IgG1) fusion (0.2 μg/well) or a human IgG1 antibody containing the CR3022 variable domain (0.05 μg/well). After incubation for 1 hour at room temperature, plates were washed three times with PBST and 200 μl 1:5000 peroxidase-conjugated anti-human IgG was added (Jackson ImmunoResearch Laboratories, Inc. Cat. #109-035-098). Plates were incubated 30 minutes at room temperature and washed three times with PBST. Finally, 70 μl Tetramethylbenzidine (TMB) (MilliporeSigma) was added and incubated 5 minutes at room temperature before quenching with 70 μl 2 M H2SO4. Absorbance at 450 nm was measured using a Biotek Synergy H1 plate reader.

### Immunogen Expression, Purification, and Calculation of Yields

Recombinant RBD immunogens were expressed in expi293F cells, as described for SPEEDesign in vitro screening above. Trimeric FL-spike ectodomain was also expressed in expi293F cells using a modified pHL-see vector. The construct contains amino acids 16-1208 of the spike protein followed by a foldon trimerization domain and a 6-His tag. This construct also contains the “2P” stabilizing mutations at K986P and V987P and mutation of the furin cleavage site (682-685 GSAS to RRAR). FL-S-2P, as used herein, represents the full-length spike protein ectodomain containing the 2P stabilizing mutations and a mutated furin cleavage site.

Cell-free supernatant was harvested after 4 days and His-tagged immunogens were purified by gravity chromatography using Ni Sepharose excel resin according to manufacturer instructions (Cytiva). Immunogens were further purified by size-exclusion chromatography using a Superdex 75 Increase 10/300 GL column (RBD immunogens) or Superose 6 Increase 10/300 GL column (trimeric FL-spike) equilibrated in 1x PBS. Fractions corresponding to trimeric FL-spike or monomeric RBD were pooled, snap frozen in liquid nitrogen, and stored at −80° C.

Transfection, expression, and purification was performed in triplicate on three separate days to calculate RBD immunogen purification yields. Each replicate consisted of a 30 mL culture, and yields were calculated by integrating the area under the monomeric peak on the Abs280 chromatogram during size-exclusion chromatography. These yields closely matched yields calculated from pooled fractions. Extinction coefficients were calculated using the ExPASy ProtParam tool.

### Antibody Expression and Purification

Antibodies for ELISAs were created by fusing the variable regions for the indicated antibody to the human IGHG*01, IGKC*01, or IGLC2*02 constant regions and cloning into the pHL-see plasmid (GenScript). The Ace2-Fc fusion was expressed from pcDNA3: pcDNA3-SACE2 (WT)-Fc (IgG1) was a gift from Erik Procko (Addgene plasmid #145163; http://n2t.net/addgene: 145163; RRID: Addgene_145163). Heavy and light chain plasmids were mixed in equal amounts and transfected into expi293F cells according to manufacturer instructions and cell-free supernatant was harvested after 4 days of expression (ThermoFisher Scientific).

Cell-free supernatant was batch incubated with protein A agarose resin (GoldBio) for 1 hour at room temperature. Resin was collected and washed with 10 column volumes (CV) protein A IgG binding buffer (ThermoFisher Scientific). Protein was eluted with 5 CV IgG elution buffer (ThermoFisher Scientific) and neutralized with 0.5 CV 1 M Tris pH 9.0. Antibodies were concentrated and buffer exchanged into PBS using an amicon centrifugal filter (MilliporeSigma). Ace2-Fc was further purified by size-exclusion chromatography using a superdex 200 increase 10/300 GL column (Cytiva) equilibrated in 1x PBS.

### Differential Scanning Fluorimetry

DSF was performed using the Protein Thermal Shift Dye Kit according to manufacturer instructions (ThermoFisher Scientific). Final reactions contained 0.125 mg/mL purified immunogen, 1× Protein Thermal Shift buffer, 1x Thermal Shift Dye, and 0.63x PBS. Fluorescence was monitored using a 7500 Fast Real-Time PCR system (ThermoFisher Scientific) as the temperature was increased from 25° C. to 95° C. at a ramp rate of 1%. Melting temperature (Tm) was calculated as the peak of the derivative of the melt curve. DSF reactions were performed in technical quadruplicate on each plate and in biological triplicate using three different protein preps on 3 separate days. Technical replicates were averaged to calculate the Tₘ for a biological replicate, and the three biological replicates were averaged to calculate the reported Tₘ.

### Crystallization

The antigen binding fragment (Fab) of P2B-2F6 was used to promote crystallization of lead immunogen 3. P2B-2F6 Fab was produced by fusing the P2B-2F6 variable region to a His-tagged human IGHG*01 C_{H}1 domain and cloning into pHL-see (GenScript). Plasmids were transfected into expi293F cells according to manufacturer instructions and cell-free supernatant was harvested after 4 days of expression (ThermoFisher Scientific). His-tagged Fab was purified from cell-free supernatant by gravity chromatography using Ni Sepharose excel resin according to manufacturer instructions (Cytiva). Fab was further purified by size-exclusion chromatography using a Superdex 75 increase 10/300 GL column (Cytiva) equilibrated in 1x PBS.

Purified Fab was mixed with purified lead immunogen 3 in a 1.5:1 ratio and incubated 30 minutes on ice. Complex was purified by size-exclusion chromatography on a Superdex 200 increase 10/300 GL column equilibrated in 10 mM Na-HEPES pH 7.4, 100 mM NaCl. Purified complex was concentrated to 18 mg/mL using an amicon centrifugal filter (MilliporeSigma) and crystal trays were setup using a mosquito crystal robot (STP Labtech). Drops contained 0.2 μl complex and 0.2 μl reservoir solution (0.2 M sodium fluoride, 20% w/v PEG 3,350). Crystals were grown by hanging-drop vapor diffusion at 18° C. for 13 days. Crystals were cryoprotected in well-solution mixed with 3 μl 100% glycerol and flash frozen in liquid nitrogen.

### Data Collection and Structure Determination

Crystal diffraction data were collected at the GM/CA 23-ID-D beamline at the Advanced Photon Source. Reflections were indexed and integrated using XDS. Data were scaled and merged using AIMLESS. The P2B-2F6/WT RBD structure (PDB: 7BWJ) was used as a starting model for rigid body refinement in PHENIX Refine. This model was then edited in COOT to incorporate the amino acid changes present in immunogen 3 and subsequent rounds of refinement and model building were performed with COOT and PHENIX Refine to produce a model with Rwork/Rfree of 21.79%/26.97%. The final model was evaluated with MolProbity, which showed good geometry, with 96.4% of the residues as Ramachandran favored and 0% outlier residues (FIG. 3). Software used in this project was curated by SBGrid.

### ELISA Analysis of Immunogen Epitopes

Nunc MaxiSorp plates (ThermoFisher Scientific) were coated with 100 μl 0.01 mg/mL purified immunogen diluted in 50 mM Na-carbonate pH 9.5. Plates were coated overnight at 4° C. then washed three times with PBST. Plates were blocked 1 hour at room temperature with 2% BSA in PBST then washed three times with PBST. 100 μl primary antibody was added to each well at the indicated concentration: Ace2-3.1 ng/ml, REGN10933, CR3022, and S309-1.5 ng/ml, and P2B-2F6-7.5 ng/ml. Primary antibody was incubated 1 hour at room temperature then plates were washed three times with PBST and 200 μl 1:5000 peroxidase-conjugated anti-human IgG was added (Jackson ImmunoResearch Laboratories, Inc. Cat. #109-035-098). Plates were incubated 30 minutes at room temperature and washed three times with PBST. Finally, 70 μl Tetramethylbenzidine (TMB) (MilliporeSigma) was added and incubated 10 minutes at room temperature before quenching with 70 μl 2 M H2SO4. Absorbance at 450 nm was measured using a Biotek Synergy H1 plate reader.

### Biolayer Interferometry

The binding affinity of purified immunogens to REGN10933 and Ace2-Fc was measured using a kinetic BLI assay using an Octet-Red96e (Sartorius). REGN10933 IgG or Ace2-Fc was buffer exchanged into HBS-EP buffer (10 mM Na-HEPES pH 7.4, 150 mM NaCl, 3 mM EDTA, 0.005% v/v P20 surfactant) using Zeba spin desalting columns (ThermoFisher Scientific). REGN10933 or Ace2-Fc was loaded onto Anti-hlgG Fc Capture (AHC) biosensors (Sartorius) over the course of 300 seconds, until reaching a signal of ˜0.6 nm. BLI pins were then immersed in immunogens 2-fold serially diluted in HBS-EP buffer (150 nM to 2.34 nM). After 300 seconds, pins were immersed in HBS-EP buffer to measure dissociation. Association rate (ka), dissociation rate (kdis), and dissociation constant (K_{D}) were globally fit using a 1:1 binding model in Data Analysis HT 12.0 (Sartorius). Three independent protein preps (biological replicates) were each measured in technical triplicate. Values reported are the average and standard deviation between biological replicates.

### Mouse Immunizations

Mouse immunogenicity studies were performed under the guidelines and approval of the Institutional Animal Care and Use Committee (IACUC) at the National Institutes of Health. Five 5-6 week old female CD-1 mice (Charles River Laboratories) per group were immunized with 10 μg antigen each. Antigen was formulated as a 1:1 ratio in Complete Freund's Adjuvant (MilliporeSigma) on day 0 and Incomplete Freund's Adjuvant (MilliporeSigma) on day 21 and 100 μl formulated antigen was delivered by intraperitoneal injection. Blood was collected on day 35 and serum was separated and stored at −80° C.

### Serum Antibody Titer ELISA

Nunc MaxiSorp plates (ThermoFisher Scientific) were coated with 100 μl 0.01 mg/mL purified trimeric FL-spike ectodomain diluted in 50 mM Na-carbonate pH 9.5. Plates were incubated overnight at 4° C. then washed three times with PBST. Plates were blocked 1 hour at room temperature with 2% BSA in PBST then washed three times with PBST. Serum was diluted in 2% BSA in PBST and 100 μl was added to each well. After 1 hour incubation at room temperature, plates were washed three times with PBST and 200 μl 1:5000 peroxidase-conjugated anti-mouse IgG was added (Jackson ImmunoResearch Laboratories, Inc. Cat. #115-035-164). Plates were incubated 30 minutes at room temperature and washed three times with PBST. Finally, 70 μl Tetramethylbenzidine (TMB) (MilliporeSigma) was added and incubated 20 minutes at room temperature before quenching with 70 μl 2 M H₂SO₄. Absorbance at 450 nm was measured using a Biotek Synergy H1 plate reader.

Pooled serum from mice immunized with WT RBD was used as a standard curve on each plate to calculate the antibody titers of individual animals in all groups. One antibody unit (AU) was defined as the dilution of the standard serum required to achieve an Abs450 value of 1. Each plate included triplicate 2-fold serial dilutions of the standard serum from 20 to 0.01 AU. Serum from each animal was diluted such that the Abs450 fell in the informative portion of the standard curve between 0.1 and 2.0. The Abs450 values for the standard curve were fit to a 4-parameter logistic curve, which was used to convert Abs450 values to AU for each individual animal. AU values for each individual animal were measured in triplicate on separate plates and the average is reported.

### RBD/Ace2 Blocking Assay

Nunc MaxiSorp plates (ThermoFisher Scientific) were coated with 100 μl 0.32 μg/mL purified WT RBD diluted in 50 mM Na-carbonate pH 9.5. Plates were incubated overnight at 4° C. then washed three times with PBST. Plates were blocked 1 hour at room temperature with 2% BSA in PBST then washed three times with PBST. Serum was diluted in 2% BSA in PBST in a 3-fold dilution series from 1:100 to 1:218,700. 50 μl serum was mixed with 10 μl 30 nM Ace2-Fc or 10 μl buffer as a background control. 50 μl serum mixture was added to RBD-coated plates and incubated 1 hour at room temperature. Plates were washed three times with PBST and 200 μl 1:30,000 peroxidase-conjugated anti-human IgG was added (Jackson ImmunoResearch Laboratories, Inc. Cat. #109-035-098). Plates were incubated 30 minutes at room temperature and washed three times with PBST. Finally, 70 μl Tetramethylbenzidine (TMB) (MilliporeSigma) was added and incubated 20 minutes at room temperature before quenching with 70 μl 2 M H₂SO₄. Absorbance at 450 nm was measured using a Biotek Synergy H₁ plate reader.

Ace2/RBD binding inhibition was calculated by first subtracting the Abs₄₅₀ values of the background controls lacking Ace2-Fc. Eight wells without serum were used to calculate the maximum signal. Inhibition was calculated using the following formula:

$$\% inhibition = 100 \times ( 1 - \frac{X}{max} )$$

where X is the Abs₄₅₀ of a well after background-subtraction and max is the average of the 8 samples without serum after background-subtraction.

% inhibition values were measured for each serum dilution in triplicate and average values were plotted in GraphPad Prism 8. Data were fit using a normalized dose response curve with a variable slope:

$$Y = 100 / ( 1 + ( \frac{IC 50}{X} ) \hat{} HillSlope )$$

where X is the serum dilution, Y is the % inhibition, and HillSlope and IC50 are calculated parameters corresponding to the slope of the curve and the dilution at which 50% inhibition occurs, respectively.

IC50 values for each animal were log-transformed and plotted, along with the geometric mean value for each group.

### Pseudoviral Neutralization

Mouse serum was diluted in a duplicate 4-fold series from 1:5 to 1:81,920. Serum was mixed 1:1 with pseudovirus containing the SARS-COV-2 spike protein and a luciferase reporter in a 96-well plate (GenScript). After 1 hour incubation at room temperature, 20,000 HEK293 cells overexpressing Ace2 were added to each well. Cells and pseudovirus were incubated at 37° C., 5% CO2 for 48 hours, after which culture medium was removed and Bio-Glo luciferase reagent (Promega) was added to the wells. Luciferase signal was measured using an EnVision plate reader and % inhibition was calculated using the following formula:

$$\% inhibition = 100 \times ( 1 - \frac{X - min}{max - min} )$$

where X is the luciferase signal, min is the average signal from duplicate wells without pseudovirus, and max is the average signal from duplicate wells without serum.

log IC50 values were calculated in the same manner described for the RBD/Ace2 blocking assay.

### EXAMPLE 7

This example demonstrates the design of a non-glycosylated RBD immunogen using deGlyc-SPEEDesign.

The SPEEDesign pipeline as disclosed herein above was modified to create a non-glycosylated RBD immunogen (deGlyc-SPEEDesign). The SPEEDesign pipeline categorizes each residue as fixed, intermediate, or deep search to define the amino acid identities sampled at each position during the computational design process. The original SPEEDesign pipeline limits the amino acids sampled at intermediate residues to those found in homologous protein sequences, thereby identifying changes compatible with the RBD and related structures. In deGlyc-SPEEDesign, this approach was modified to leverage the available deep mutational scanning data. This modified approach limits the computational search at intermediate residues to only those amino acid changes that have positive effects on expression in yeast. However, this deep mutational scanning data was collected on glycosylated proteins and does not identify changes that will compensate for the removal of the glycan. Therefore, the NxS/T motif and all residues that contact the glycan was defined as deep search, allowing them to sample all amino acids, except cysteine (FIG. 10B). Finally, fixed amino acids were defined to maintain neutralizing epitopes while allowing the computational design process to find an optimally stabilized solution (FIG. 10B). The most potent neutralizing antibodies are those that block Ace2 binding; therefore, all amino acids comprising the Ace2 binding site were ensured to be unchanged in the design process. A second design strategy was also used in which the epitopes of all known antibodies that block Ace2 binding were unchanged.

A total of 20,000 deGlyc-SPEEDesign decoys were produced by the two design strategies, and 24 sequences were selected to sample the top scoring decoys in a sparse-matrix fashion (FIG. 10C). These 24 sequences were synthesized and expressed in expi293F cell culture, and the cell-free supernatant was screened by ELISA for the presence of designed immunogens that retain binding to Ace2 and the blocking antibody REGN10933. One non-glycosylated RBD immunogen (decoy 4, corresponding to SEQ ID NO: 7 shown in FIG. 3) bound both ELISA probes with reactivity far greater than WT RBD. The ELISA signal is comparable to optimized glycosylated immunogens previously reported (leads 1-5), suggesting that this non-glycosylated immunogen can be expressed at high levels with neutralizing epitopes intact.

### EXAMPLE 8

This example demonstrates that expression and stability of non-glycosylated RBD immunogen exceeds WT RBD.

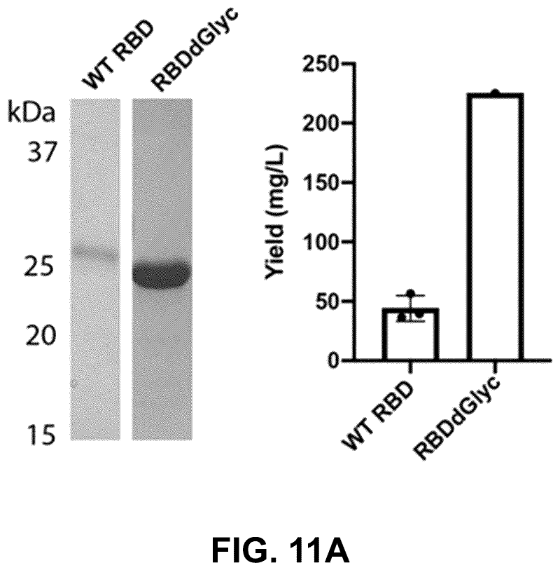

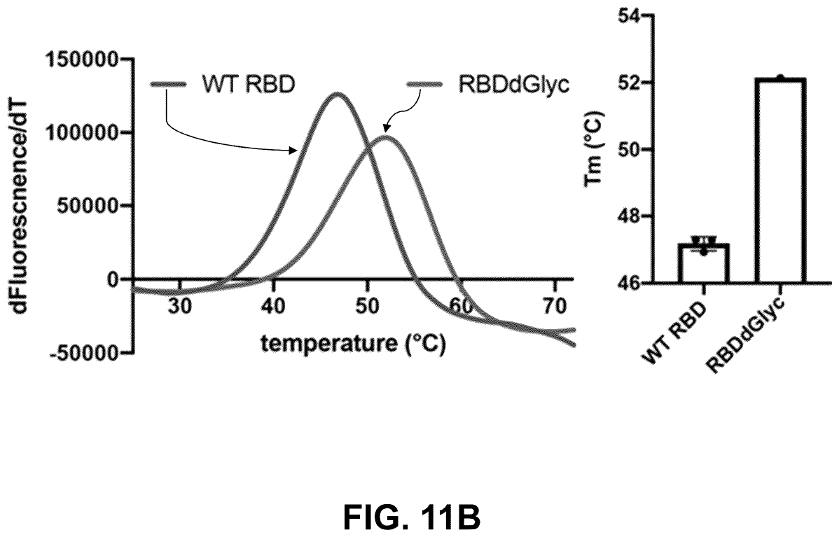

Recombinant non-glycosylated RBD immunogen (RBDdGlyc) was expressed and purified from expi293F cells to validate the biophysical characteristics of the purified protein. Purified RBDdGlyc is homogenous by SDS-PAGE and migrates at a molecular weight consistent with a non-glycosylated RBD that is smaller than the glycosylated WT RBD (FIG. 11A). Furthermore, the final purification yields for RBDdGlyc are over 200 mg/L, a greater than 5-fold increase over WT RBD. The thermostability of RBDdGlyc was measured by differential scanning fluorimetry (DSF) to determine if the stabilizing mutations fully compensated for removal of the glycan. RBDdGlyc has a melting temperature of 52° C. which is 5° C. higher than WT RBD and similar to the thermostabilized glycosylated RBD immunogens previously reported (FIG. 11B). Thus, RBDdGlyc is a homogenous immunogen with improved expression and stability.

### EXAMPLE 9

This example demonstrates that RBDdGlyc can be assembled into single component nanoparticles.

In some embodiments, immunogenicity of an RBD antigen is significantly enhanced by nanoparticle display. This concept has been demonstrated using two-component nanoparticle platforms where a purified RBD subunit is mixed with a separately purified nanoparticle subunit. In some embodiments, a nanoparticle comprises at least one protein having at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof. In some embodiments, a nanoparticle comprises at least one partial coronavirus spike protein comprising a receptor binding domain (RBD), wherein the RBD has at least 90% sequence homology with a sequence selected from the group consisting of SEQ ID NO: 2, SEQ ID NO: 3, SEQ ID NO: 4, SEQ ID NO: 5, SEQ ID NO: 6, and any combination thereof.

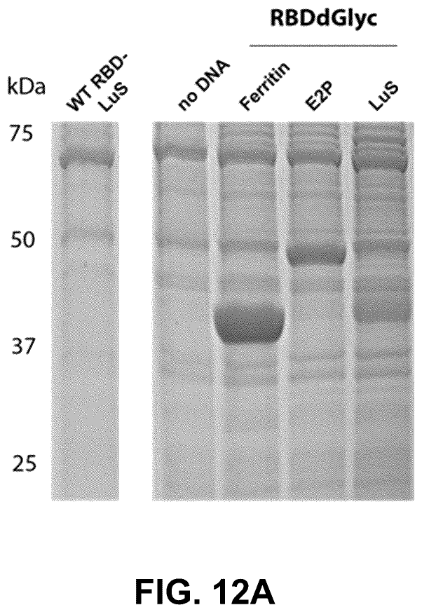

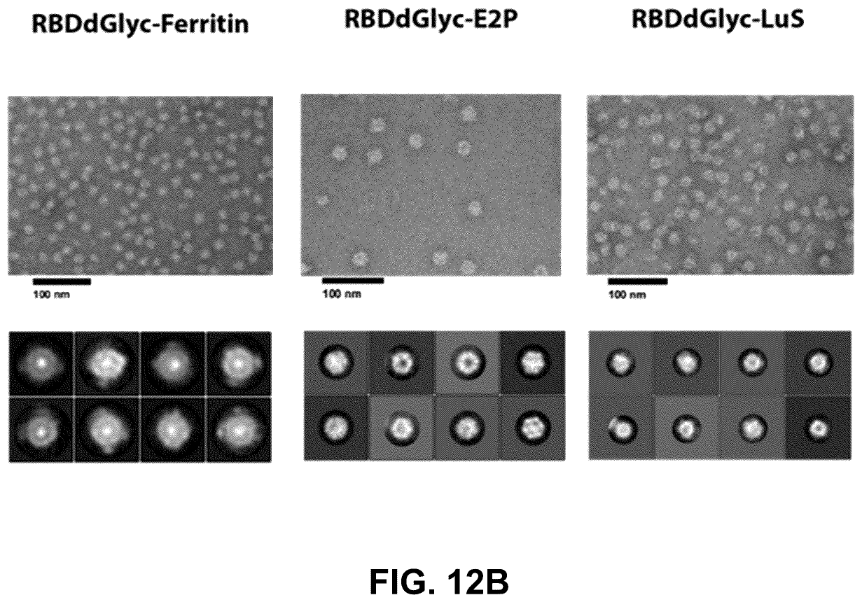

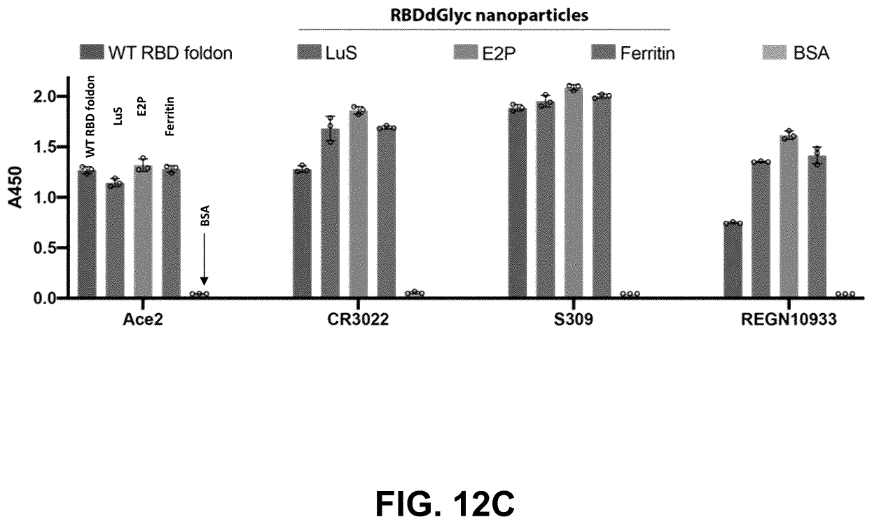

Two-component platforms, however, require optimization of multiple purification steps and an assembly step, which complicate the manufacturing process and reduce the final yield of the desired product. Single-component nanoparticles do not suffer from these complications. However, glycosylated RBD antigens cannot be expressed as nanoparticle fusions (FIG. 12A). While it was attempted to produce fusions of several different glycosylated RBD immunogens with several different nanoparticle subunits, none of these single-component nanoparticles could be expressed at high levels (data not shown).

As disclosed herein, glycosylated RBD prevents nanoparticle expression and assembly due to steric clash of bulky glycans upon high density display on a nanoparticle, and non-glycosylated RBD more readily assemble into nanoparticles. In some embodiments, a nanoparticle comprises at least one non-glycosylated receptor binding domain (RBD) recombinant protein having at least 85% sequence homology with SEQ ID NO: 7. In some embodiments, a nanoparticle comprises at least one partial coronavirus spike protein comprising a non-glycosylated receptor binding domain (RBD), wherein the non-glycosylated RBD has at least 85% sequence homology with SEQ ID NO: 7. Indeed, RBDdGlyc is highly expressed as a fusion with all nanoparticle platforms tested (FIG. 12A), including ferritin, dihydrolipoyl acetyltransferase (E2P), and Lumazine Synthase (LuS). Those skilled in the art will recognize that RBDdGlyc will be compatible any other suitable nanoparticle platform, e.g., hepatitis B surface antigen (HBsAg), human papilloma virus protein LI (HPV L1), etc. These fusion proteins elute from a size-exclusion column at a volume consistent with the formation of large complexes, and negative stain electron microscopy confirms the formation of homogenous nanoparticles (FIG. 12B). Finally, the neutralizing epitopes on RBDdGlyc are accessible in the assembled nanoparticles suggesting that these RBD nanoparticle vaccine candidates are capable of eliciting strong neutralizing responses against SARS-COV-2 (FIG. 12C).

The novel non-glycosylated RBD nanoparticle antigens (RBDdGlyc) disclosed herein elicit blocking antibody titers in mice comparable to a gold-standard full-length spike-2P (FL-S-2P) antigen. Creation of a non-glycosylated RBD (see SEQ ID NO: 7) facilitated production of single-component RBD nanoparticles (FIG. 12A-C). Subsequently, mice immunized with these nanoparticles demonstrated that blocking antibody titers were within 2-3 fold of those elicited by FL-S-2P. FL-S-2P is comparable to the antigen used in all currently authorized vaccines, such that the novel nanoparticle antigen of the present disclosure performs comparably to leading antigens, with improved manufacturing and distribution benefits as described herein elsewhere.

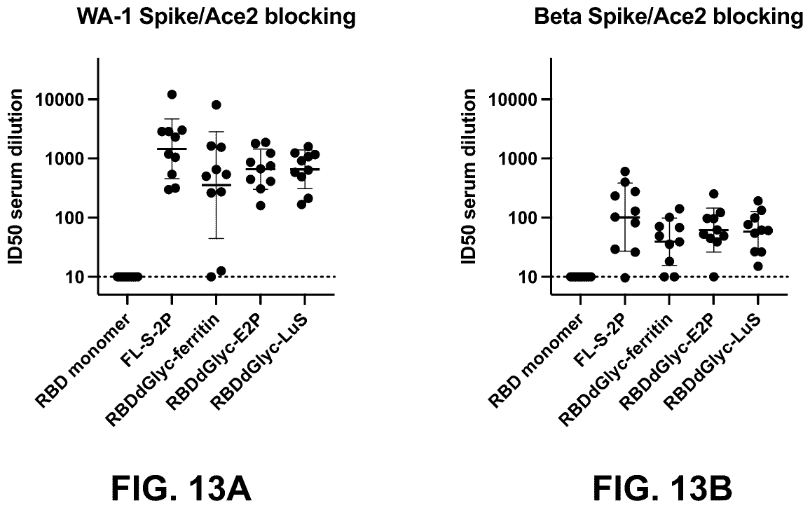

Nanoparticles elicited immune responses similar to FL-S-2P (FIG. 13A-B). Immunized mice with the novel self-assembling non-glycosylated RBD nanoparticles described herein (RBDdGlyc) were compared the response to immunization with monomeric RBD and a gold-standard FL-S-2P antigen (FIG. 13A and FIG. 13B). The 60-copy nanoparticles (RBDdGlyc-E2P and -LuS) elicited significantly higher titers of blocking antibodies than monomeric RBD against both the WA-1 and Beta viral variants. The blocking titers elicited by the nanoparticles were comparable to those elicited by the FL-S-2P.

### Conclusions

The present disclosure describes both novel compositions of matter that produce better neutralizing responses than current Spike-based immunogens, as well as a novel methodology for enhanced antigen design (SPEEDesign). With respect to the aspect of novel compositions of matter, designed sequences are disclosed herein based on the Spike receptor binding domain (RBD), including three exemplary embodiments of enhanced RBD designs for use alone or in combination with full length Spike, and including one exemplary embodiment of enhanced RBD design for nanoparticle use. These sequences disclosed herein are unique and not found in nature, and therefore constitute novel compositions of matter. The associated immunogens disclosed herein retain structured neutralizing epitopes, as supported by preclinical animal studies with these immunogens resulting in neutralizing responses significantly greater than current antigens. With respect to the novel methodology aspect (SPEEDesign), exemplary embodiments included successful application of the methodology to over six antigens in the context of both RBD and NTD domains alone or incorporated into the full-length spike protein, thus proving the methodology generally applicable and advantageous for vaccine design.

The results disclosed herein demonstrate that novel RBD immunogens created by SPEEDesign induce a greater protective antibody response than existing vaccine candidates, in addition to their substantially improved biophysical characteristics. These monomeric RBD immunogens are expected to be effective vaccines in their own right, and further may be optimized as needed using methods such as foldon trimerization, multimerization, and/or nanoparticle display. For example, development of multimeric recombinant antigens include foldon trimerization, incorporation of amino acid changes into the FL trimeric spike, and nanoparticle display. The multivalent optimized immunogens are expected to produce even greater improvements over existing vaccine candidates. In the context of FL-spike, the mutations confer additional benefits by promoting the RBD-up conformation in which neutralizing epitopes are exposed (FIG. 9A-B). As immunogens are adapted to all vaccine platforms, the immunogens disclosed herein are adaptable by making select amino acid changes to the RBD sequence within the full-length Spike or within RBD-only vaccines. In some embodiments, these amino acid changes increase RBD stability, enhance the protective immune response, and lengthen the lifetime of protection conferred by a SARS-COV-2 vaccine, regardless of the platform. In addition to the enhanced protection, the improvements in stability and yield suggest that recombinant RBD immunogens are capable of being easily manufactured and distributed to meet the global need for a SARS-COV-2 vaccine.

Development of the novel compositions of matter disclosed herein included designed sequences based on the Spike receptor binding domain (RBD) of the SARS-COV-2 virus. These sequences are unique and not found in nature. These sequences produce recombinant-designed immunogens that: have better neutralizing immune responses, have higher stability, and are able to be expressed at much greater yields than the wild type RBD protein. These immunogens retain structured neutralizing epitopes, such that animal studies with these immunogens result in neutralizing responses greater than wildtype RBD and greater than conventional antigens in current clinical trials. Further development of the disclosed immunogens using nanoparticles is likely to enhance the response even further.

The present disclosure enables a SARS-COV-2 vaccine with improved breadth and duration of protection. Preclinical data of this disclosure supports that FL-Spike and RBD recombinant protein vaccines can be improved by incorporating the novel amino acid changes described herein. Consequently, other vaccine platforms (e.g. mRNA, viral vectored, inactivated virus, etc.) can also be improved by the disclosed amino acid changes. Accordingly, all SARS-CoV-2 vaccines (e.g., including all currently authorized vaccines) that utilize an antigen containing the RBD, including FL-spike, may be improved by the subject matter of the present disclosure.

Further, there is an additional market opportunity for a low-cost, easily manufactured alternative to the currently authorized vaccines. Global supply remains inadequate and will be exacerbated by the diversion of supply to boosters. The novel RBD nanoparticle described herein is smaller and easier to manufacture than the FL-Spike protein, and it is more immunogenic than the naturally occurring RBD sequence. Preclinical data of this disclosure demonstrates the novel RBD nanoparticle elicits functional antibodies at levels comparable to the more complex FL-Spike antigen, positioning this nanoparticle antigen as an accessible and effective alternative to conventional treatments. The stabilized antigens developed herein are additionally suitable for immunoassay applications. While conventional immunoassays generally involve immobilization of antigens and may require long-term storage before field use, the thermostabilized RBD antigens of the present disclosure are favorable for more reproducible and flexible immunoassays. The antigen design method (SPEEDesign) described herein is enabled for vaccine manufacturers developing new vaccines or improving established vaccines. This method also enables improvement of antigens for immunoassays.

All people would benefit from a COVID vaccine. While leading vaccine candidates may confer short-lived protection and require frequent dosing, high-yield recombinant immunogens such as those disclosed herein can significantly reduce costs. Current ultra-cold chain requirements for leading vaccines limit distribution, however, highly stable recombinant immunogens such as those described herein can alleviate cold-chain requirements. Further, leading vaccines may not protect against infection and spread, thus potentially increasing virus prevalence and risk to unvaccinated populations. The higher neutralizing titers conferred by immunogens disclosed herein may likely prevent infection, and these stabilized immunogens may be amenable to more protective methods of immunization (e.g. intranasal vaccination). While FL-spike vaccines have imperfect safety profiles (especially as antibody protection wanes), the disclosed RBD immunogens reduce potentially pathogenic non-neutralizing antibodies, thus improving the safety profile for RBD immunogen-based vaccines, especially for low-risk populations like children.

The teaching and content of the following references are hereby incorporated by reference.
Adams P. D. et al., PHENIX: a comprehensive Python-based system for macromolecular structure solution. Acta Crystallogr D Biol Crystallogr 66, 213-221 (2010).
Agrawal, A. S. et al. Immunization with inactivated Middle East Respiratory Syndrome coronavirus vaccine leads to lung immunopathology on challenge with live virus. Human vaccines & immunotherapeutics 12 (9), 2351-6 (2016).
Andreano, E. et al., Extremely potent human monoclonal antibodies from convalescent Covid-19 patients. bioRxiv (2020).
Aricescu A. R., W. Lu, E. Y. Jones, A time- and cost-efficient system for high-level protein production in mammalian cells. Acta Crystallogr D Biol Crystallogr 62, 1243-1250 (2006).
Barnes, C. O. et al., SARS-COV-2 neutralizing antibody structures inform therapeutic strategies. Nature, 1-9 (2020).
Bolles, M. et al. A Double-Inactivated Severe Acute Respiratory Syndrome Coronavirus Vaccine Provides Incomplete Protection in Mice and Induces Increased Eosinophilic Proinflammatory Pulmonary Response upon Challenge. Journal of Virology 85 (23), 12201-12215 (2011).
Bos, R. et al., Ad26 vector-based COVID-19 vaccine encoding a prefusion-stabilized SARS-COV-2 Spike immunogen induces potent humoral and cellular immune responses. Npj Vaccines 5, 91 (2020).
Chan K. K. et al., Engineering human ACE2 to optimize binding to the spike protein of SARS coronavirus 2. Science 369, 1261-1265 (2020).
Chen V. B. et al., MolProbity: all-atom structure validation for macromolecular crystallography. Acta Crystallogr D Biol Crystallogr 66, 12-21 (2010).
Corbett, K. S. et al., SARS-COV-2 mRNA vaccine design enabled by prototype pathogen preparedness. Nature 586, 567-571 (2020).
Dai, L. et al., A universal design of betacoronavirus vaccines against COVID-19, MERS and SARS. Cell 182 (3), 722-733.e711 (2020).
Emsley P. et al., Features and development of Coot. Acta Crystallogr D Biol Crystallogr 66, 486-501 (2010).
Evans P. R., G. N. Murshudov, How good are my data and what is the resolution? Acta Crystallogr D Biol Crystallogr 69, 1204-1214 (2013).
Fleishman et al., Automated Structure- and Sequence-Based Design of Proteins for High Bacterial Expression and Stability, Mol. Cell, 2016, 63 (2), 337-346.
Folegatti, P. M. et al. Safety and immunogenicity of the ChAd0x1 nCOV-19 vaccine against SARS-COV-2: a preliminary report of a phase 1/2, single-blind, randomised controlled trial. Lancet 396 (10249), 467-478 (2020).
Gasteiger E. et al., ExPASy: The proteomics server for in-depth protein knowledge and analysis. Nucleic Acids Res 31, 3784-3788 (2003).
Goldenzweig A. et al., Automated Structure- and Sequence-Based Design of Proteins for High Bacterial Expression and Stability. Mol Cell 63 (2), 337-346 (2016).
Hansen, J. et al., Studies in humanized mice and convalescent humans yield a SARS-CoV-2 antibody cocktail. Sci New York N Y, eabd0827 (2020).
He, L. et al. Self-assembling nanoparticles presenting receptor binding domain and stabilized spike as next-generation COVID-19 vaccines. bioRxiv, 2020.09.14.296715 (2020).
Hsieh, C.-L. et al., Structure-based design of prefusion-stabilized SARS-COV-2 spikes. Science, eabd0826 (2020).
Hwang W. C. et al., Structural Basis of Neutralization by a Human Anti-severe Acute Respiratory Syndrome Spike Protein Antibody, 80R. J Biol Chem 281 (45), 34610-34616 (2006).
Iwasaki, A. and Yang, Y. The potential danger of suboptimal antibody responses in COVID-19. Nature Reviews Immunology, 1-3 (2020).
Jackson, L. A. et al., An mRNA Vaccine against SARS-COV-2-Preliminary Report. N Engl J Med 383, 1920-1931 (2020).
Ju, B. et al., Human neutralizing antibodies elicited by SARS-COV-2 infection. Nature 584, 115-119 (2020).
Kabsch W., Xds. Acta Crystallogr D Biol Crystallogr 66, 125-132 (2010).
Keech, C. et al., Phase 1-2 Trial of a SARS-COV-2 Recombinant Spike Protein Nanoparticle Vaccine. N Engl J Med 383, 2320-2332 (2020).
Kirchdoerfer, R. N. et al. Stabilized coronavirus spikes are resistant to conformational changes induced by receptor recognition or proteolysis. Scientific Reports 8 (1), 15701 (2018).
Lan J. et al., Structure of the SARS-COV-2 spike receptor binding domain bound to the ACE2 receptor. Nature 581, 215-220 (2020).
Li W., A. Godzik, Cd-hit: a fast program for clustering and comparing large sets of protein or nucleotide sequences. Bioinformatics 22, 1658-1659 (2006).
Liu, L. et al. Anti-spike IgG causes severe acute lung injury by skewing macrophage responses during acute SARS-COV infection. JCI insight 4 (4), e123158 (2019).
Liu, L. et al., Potent neutralizing antibodies directed to multiple epitopes on SARS-CoV-2 spike. Nature, 1-10 (2020).
Meulen J. t. et al., Human Monoclonal Antibody Combination against SARS Coronavirus: Synergy and Coverage of Escape Mutants. Plos Med 3 (7), e237 (2006).
Morin A. et al., Collaboration gets the most out of software. Elife 2, e01456 (2013). Winn M. D. et al., Overview of the CCP4 suite and current developments. Acta Crystallogr D Biol Crystallogr 67, 235-242 (2011).
Pak J. E. et al., Structural Insights into Immune Recognition of the Severe Acute Respiratory Syndrome Coronavirus S Protein Receptor Binding Domain. J Mol Biol 388 (4), 815-823 (2009).
Pallesen, J. et al., Immunogenicity and structures of a rationally designed prefusion MERS-COV spike antigen. P Natl Acad Sci Usa 114 (35), E7348-E7357 (2017).
Prabakaran P. et al., Structure of Severe Acute Respiratory Syndrome Coronavirus Receptor binding Domain Complexed with Neutralizing Antibody. J Biol Chem 281 (23), 15829-15836 (2006).
Sadoff, J. et al. Safety and immunogenicity of the Ad26.COV2.S COVID-19 vaccine candidate: interim results of a phase 1/2a, double-blind, randomized, placebo-controlled trial. medRxiv (2020).
S. L. 2010, The PyMOL Molecular Graphics System. Version 2.4.
Starr, T. N. et al. Deep Mutational Scanning of SARS-COV-2 Receptor Binding Domain Reveals Constraints on Folding and ACE2 Binding. Cell 182 (5), 1295-1310 e20 (2020).
Sui J. et al., Potent neutralization of severe acute respiratory syndrome (SARS) coronavirus by a human mAb to S1 protein that blocks receptor association. P Natl Acad Sci Usa 101 (8), 2536-2541 (2004).
Tan, T. K. et al., A COVID-19 vaccine candidate using SpyCatcher multimerization of the SARS-COV-2 spike protein receptor binding domain induces potent neutralising antibody responses. bioRxiv (2020).
Tseng, C.-T. et al. Immunization with SARS Coronavirus Vaccines Leads to Pulmonary Immunopathology on Challenge with the SARS Virus. PLOS ONE 7 (4), e35421 (2012).
Walls A. C. et al., Unexpected Receptor Functional Mimicry Elucidates Activation of Coronavirus Fusion. Cell 176, 1026-1039.e1015 (2019).
Walls, A. C. et al. Elicitation of potent neutralizing antibody responses by designed protein nanoparticle vaccines for SARS-COV-2. bioRxiv, 2020.08.11.247395 (2020).
Walls, A. C. et al., Elicitation of potent neutralizing antibody responses by designed protein nanoparticle vaccines for SARS-COV-2. bioRxiv, 2020.2008.2011.247395 (2020).
Walsh, E. E. et al., Safety and Immunogenicity of Two RNA-Based Covid-19 Vaccine Candidates. N Engl J Med 383, 2439-2450 (2020).
Wang, Q. et al. Immunodominant SARS Coronavirus Epitopes in Humans Elicited both Enhancing and Neutralizing Effects on Infection in Non-human Primates. ACS Infectious Diseases 2 (5), 361-376 (2016).
Watanabe, Y. et al. Site-specific glycan analysis of the SARS-COV-2 spike. Science 369 (6501), 330-333 (2020).
World Health Organization (WHO), Draft landscape of Covid-19 candidate vaccines. https://www.who.int/blueprint/priority-diseases/key-action/novel-coronavirus-landscape-ncov.pdf (2020).
Wrapp D. et al., Cryo-EM structure of the 2019-nCOV spike in the prefusion conformation. Science 367, 1260-1263 (2020).
Wrapp D. et al., Structural Basis for Potent Neutralization of Betacoronaviruses by Single-Domain Camelid Antibodies. Cell 181, 1004-1015.e1015 (2020).
Wu, K. et al., mRNA-1273 vaccine induces neutralizing antibodies against spike mutants from global SARS-COV-2 variants. bioRxiv (2021).
Yang, J. et al., A vaccine targeting the RBD of the S protein of SARS-COV-2 induces protective immunity. Nature 586, 572-577 (2020).
Yao, H. et al. Molecular Architecture of the SARS-COV-2 Virus. Cell 183 (3), 730-738 e13 (2020).
Yuan M. et al., A highly conserved cryptic epitope in the receptor binding domains of SARS-COV-2 and SARS-COV. Sci New York N Y, eabb7269 (2020).
Zhang, Y. et al. Site-specific N-glycosylation Characterization of Recombinant SARS-COV-2 Spike Proteins. Mol Cell Proteomics (2020).
Zhou T. et al., Cryo-EM Structures of SARS-COV-2 Spike without and with ACE2 Reveal a pH-Dependent Switch to Mediate Endosomal Positioning of Receptor binding Domains. Cell Host Microbe 28, 867-879 e865 (2020).

1. A protein comprising an amino acid sequence selected from the group consisting of:
i) an amino acid sequence having at least 90% sequence homology with over the entire length of a sequence selected from the group consisting of SEQ ID NO: 2, SEQ ID NO: 3, SEQ ID NO: 4, SEQ ID NO: 5, and SEQ ID NO: 6; and,
ii) an amino acid sequence having at least 85% sequence homology over the entire length of SEQ ID NO:7;
wherein the amino acid sequence comprises an amino acid having a larger side chain than alanine at the position corresponding to position 31 of SEQ ID NO:1, a polar or charged amino acid at the position corresponding to position 136 of SEQ ID NO:1, or an amino acid more hydrophilic than histidine at the position corresponding to position 187 of SEQ ID NO:1.

2-104. (canceled)

105. The protein of claim 1, wherein the amino acid at a position corresponding to position 31 of SEQ ID NO:1 is selected from the group comprising Tyr, Phe, Trp, Ile, Leu, Met, Val, Lys, Arg, and His.

106. The protein of claim 105, wherein the amino acid at the position corresponding to position 31 of SEQ ID NO: 1 is selected from the group comprising Tyr, Phe, and Trp.

107. The protein of claim 1, wherein the amino acid at a position corresponding to position 136 of SEQ ID NO:1 is selected from the group consisting of Thr, Ser, Asn, Gln, Glu, Asp, His, Arg, and Lys.

108. The protein of claim 107, wherein the amino acid at the position corresponding to position 136 of SEQ ID NO:1 is selected from the group consisting of Thr, Ser, Asn, Gln, Glu, Asp, Arg, and Lys.

109. The protein of claim 1, wherein the amino acid at a position corresponding to position 187 of SEQ ID NO: 1 is selected from the group consisting of Asp, Glu, Lys, Arg, Gln, and Asn.

110. The protein of claim 109, wherein the amino acid at the position corresponding to position 187 of SEQ ID NO: 1 is selected from the group consisting of Asp and Glu.

111. The protein of claim 1, wherein the amino acid sequence comprises a tyrosine, a phenylalanine or a tryptophan at the position corresponding to position 31 of SEQ ID NO:1, a threonine at the position corresponding to position 136 of SEQ ID NO:1, and an aspartic acid at the position corresponding to position 187 of SEQ ID NO:1.

112. The protein of claim 1, wherein the amino acid at a position corresponding to position 190 of SEQ ID NO:1 is a proline.

113. The protein of claim 1, wherein the amino acid sequence is selected from the group consisting of:
a. an amino acid sequence at least 90% identical to SEQ ID NO:2, wherein the amino acid sequence comprises a methionine at the position corresponding to position 1 of SEQ ID NO:2, a proline at the position corresponding to position 16 of SEQ ID NO:2, a tyrosine at the position corresponding to position 30 of SEQ IDNO: 2, a tyrosine at the position corresponding to position 31 of SEQ ID NO:2, a glutamine at the position corresponding to position 62 of SEQ ID NO:2, a phenylalanine at the position corresponding to position 64 of SEQ ID NO:2, a threonine at the position corresponding to position 136 of SEQ ID NO:2, an aspartic acid at the position corresponding to position 187 of SEQ IDNO: 2, or a proline at the position corresponding to position 190 of SEQ IDNO: 2;
b. an amino acid sequence at least 90% identical to SEQ ID NO:3, wherein the amino acid sequence comprises a glutamine at the position corresponding to position 30 of SEQ ID NO:3, a phenylalanine at the position corresponding to position 31 of SEQ ID NO:3, a lysine at the position corresponding to position 35 of SEQ ID NO:3, a glutamine at the position corresponding to position 62 of SEQ ID NO:3, a threonine at the position corresponding to position 136 of SEQ ID NO: 3, a threonine at the position corresponding to position 182 of SEQ ID NO:3, or an aspartic acid at the position corresponding to position 187 of NO: 3;
c. an amino acid sequence at least 90% identical to SEQ ID NO:4, wherein the amino acid sequence comprises a lysine at the position corresponding to position 1 of SEQ ID NO:4, a tyrosine at the position corresponding to position 31 of SEQ ID NO:4, a lysine at the position corresponding to position 56 of SEQ ID NO:4, a tyrosine at the position corresponding to position 62 of SEQ ID NO:4, a threonine at the position corresponding to position 136 of SEQ ID NO: 4, a threonine at the position corresponding to position 182 of SEQ ID NO:4, an aspartic acid at the position corresponding to position 187 of NO: 4, or a proline at the position corresponding to position 190 of SEQ ID NO:4;
d. an amino acid sequence at least 90% identical to SEQ ID NO:5, wherein the amino acid sequence comprises a lysine at the position corresponding to position 30 of SEQ ID NO:5, a tyrosine at the position corresponding to position 31 of SEQ ID NO:5, an alanine at the position corresponding to position 35 of SEQ ID NO:5, a glutamic acid at the position corresponding to position 62 of SEQ ID NO:5, a tyrosine at the position corresponding to position 67 of SEQ ID NO: 5, a valine at the position corresponding to position 70 of SEQ ID NO:5, a threonine at the position corresponding to position 136 of SEQ ID NO:5, a threonine at the position corresponding to position 182 of SEQ ID NO:5, a lysine at the position corresponding to position 186 of SEQ ID NO:5, or an aspartic acid at the position corresponding to position 187 of NO: 5;
e. an amino acid sequence at least 90% identical to SEQ ID NO:6, wherein the amino acid sequence comprises a proline at the position corresponding to position 16 of SEQ ID NO:6, an alanine at the position corresponding to position 17 of SEQ ID NO:6, a phenylalanine at the position corresponding to position 31 of SEQ ID NO:6, a phenylalanine at the position corresponding to position 33 of SEQ ID NO:6, a lysine at the position corresponding to position 35 of SEQ ID NO:6, a glutamine at the position corresponding to position 130 of SEQ ID NO:6, a threonine at the position corresponding to position 136 of SEQ ID NO:6, a glutamic acid at the position corresponding to position 138 of SEQ ID NO:6, an asparagine at the position corresponding to position 182 of SEQ ID NO:6, an aspartic acid at the position corresponding to position 187 of NO: 6, or a proline at the position corresponding to position 190 of SEQ ID NO:6; and,
f. an amino acid sequence at least 85% identical to SEQ ID NO:7, wherein the amino acid sequence comprises
a proline at the position corresponding to position 3 of SEQ ID NO:7,
a glutamic acid at the position corresponding to position 7 of SEQ ID NO:7,
an isoleucine at the position corresponding to position 9 of SEQ ID NO:7,
a histidine at the position corresponding to position 11 of SEQ ID NO:7,
a glutamic acid at the position corresponding to position 13 of SEQ ID NO:7,
a glutamine at the position corresponding to position 22 of SEQ ID NO:7,
a glutamine at the position corresponding to position 25 of SEQ ID NO:7,
an aspartic acid at the position corresponding to position 28 of SEQ ID NO:7,
a tryptophan at the position corresponding to position 31 of SEQ ID NO:7,
a phenylalanine at the position corresponding to position 33 of SEQ ID NO:7,
a leucine at the position corresponding to position 35 of SEQ ID NO:7,
a leucine at the position corresponding to position 37 of SEQ ID NO:7,
a lysine at the position corresponding to position 40 of SEQ ID NO:7,
a lysine at the position corresponding to position 53 of SEQ ID NO:7,
a glutamine at the position corresponding to position 54 of SEQ ID NO:7,
a glutamine at the position corresponding to position 76 of SEQ ID NO:7,
an aspartic acid at the position corresponding to position 108 of SEQ ID NO:7,
an aspartic acid at the position corresponding to position 118 of SEQ ID NO:7,
an aspartic acid at the position corresponding to position 127 of SEQ ID NO:7,
a lysine at the position corresponding to position 128 of SEQ ID NO:7,
a tyrosine at the position corresponding to position 132 of SEQ ID NO:7,
a threonine at the position corresponding to position 136 of SEQ ID NO:7,
an aspartic at the position corresponding to position 145 of SEQ ID NO:7,
an asparagine at the position corresponding to position 158 of SEQ ID NO:7,
a threonine at the position corresponding to position 182 of SEQ ID NO:7,
a glutamine at the position corresponding to position 184 of SEQ ID NO:7, and,
an aspartic acid at the position corresponding to position 187 of SEQ ID NO:7.

114. The protein of claim 1, wherein the amino acid sequence comprises SEQ ID NO: 2. SEQ ID NO:3, SEQ ID NO:4, SEQ ID NO:5, SEQ ID NO:6 or SEQ ID NO:7.

115. The protein of claim 1, wherein the protein is a SARS-COV-2 spike protein.

116. A nucleic acid molecule encoding the protein of claim 1.

117. A nanoparticle comprising the protein of claim 1.

118. A method of vaccinating an individual against SARS-COV-2, comprising administering to the individual a protein comprising an amino acid sequence selected from the group consisting of:
i) an amino acid sequence having at least 90% sequence homology over the entire length of a sequence selected from the group consisting of SEQ ID NO: 2, SEQ ID NO: 3, SEQ ID NO: 4, SEQ ID NO: 5, and SEQ ID NO: 6; and,
ii) an amino acid sequence having at least 85% sequence homology over the entire length of SEQ ID NO:7;
wherein the amino acid sequence comprises an amino acid having a larger side chain than alanine at the position corresponding to position 31 of SEQ ID NO: 1, a polar or charged amino acid at the position corresponding to position 136 of SEQ ID NO:1, or an amino acid more hydrophilic than histidine at the position corresponding to position 187 of SEQ ID NO:1.

119. A method for identifying at least one vaccine candidate, the method comprising:
determining a computational search depth for each amino acid position of a protein sequence of a target antigen as one of fixed, intermediate, or deep search;
performing a computational search for each amino acid position,
wherein a fixed depth is defined as leaving a residue of the protein sequence unchanged,
wherein an intermediate depth is defined as i) leaving a residue of the protein sequence unchanged, or ii) sampling from a defined pool of amino acids; and,
wherein a deep search depth is defined as allowing sampling of all amino acids, optionally excepting cysteine;
clustering high-scoring decoy sequences resulting from the computational search based on sequence similarity; and,
screening at least one decoy sequence from each cluster in vitro.

120. The method of claim 119, wherein sampling from a defined pool of amino acids comprises allowing sampling of all amino acids.

121. The method of claim 119, wherein sampling from a defined pool of amino acids comprises allowing sampling of amino acids found in proteins with similar sequences based on evolutionary constraints.

122. The method of claim 119, wherein sampling from a defined pool of amino acids comprises allowing sampling of amino acids that do not decrease expression of the protein.
